(19) United States
(12) Patent Application Publication
(10) Pub. No.: US 2006/0024304 A1
(43) Pub. Date: Feb. 2, 2006

(54) Immunoglobulins comprising predominantly a Man5GlcNAc2 glycoform

(76) Inventors: Tillman U. Gerngross, Hanover, NH (US); Huijuan Li, Lebanon, NH (US); Stefan Wildt, Lebanon, NH (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 11/187,196

(22) Filed: Jul. 21, 2005

Related U.S. Application Data
(63) Continuation-in-part of application No. 10/371,877, filed on Feb. 20, 2003.
Application No. 10/371,877 is a continuation-in-part of application No. 09/892,591, filed on Jun. 27, 2001.
(60) Provisional application No. 60/589,926, filed on Jul. 21, 2004.
(60) Provisional application No. 60/589,979, filed on Jul. 21, 2004.
(60) Provisional application No. 60/214,358, filed on Jun. 28, 2000.
(60) Provisional application No. 60/215,638, filed on Jun. 30, 2000.
(60) Provisional application No. 60/279,997, filed on Mar. 30, 2001.

(51) Int. Cl. A61K 39/395; C07H 21/04; C12P 21/06; C40B 40/10

(52) U.S. Cl. 424/143.1; 435/69.1; 435/320.1; 435/329; 530/387.3; 536/23.53

(57) ABSTRACT

The present invention relates to immunoglobulin glycoprotein compositions having predominant N-glycan structures on an immunoglobulin glycoprotein which confer a specific effector function. Additionally, the present invention relates to pharmaceutical compositions comprising an antibody having a particular enriched N-glycan structure, wherein said N-glycan structure is Man₅GlcNAc₂.

### RELATED APPLICATIONS

This application claims the benefit of U.S. Provisional Application No. 60/589,926 filed Jul. 21, 2004 and U.S. Provisional Application No. 60/589,979, filed Jul. 21, 2004; and is a continuation-in-part of U.S. application Ser. No. 10/371,877, filed Feb. 20, 2003, which is a CIP of U.S. application Ser. No. 09/892,591, filed Jun. 27, 2001, which claims the benefit of U.S. Provisional Application Ser. No. 60/214,358, filed Jun. 28, 2000, U.S. Provisional Application No. 60/215,638, filed Jun. 30, 2000, and U.S. Provisional Application No. 60/279,997, filed Mar. 30, 2001. Each of the above cited applications is incorporated herein by reference in its entirety.

### FIELD OF THE INVENTION

The present invention relates to compositions and methods for producing glycoproteins having specific N-linked glycosylation patterns. Particularly, the present invention relates to compositions of immunoglobulin glycoproteins comprising a plurality of N-glycans having specific N-glycan structures, and more particularly, to compositions comprising immunoglobulin glycoproteins wherein within the plurality there are one or more predominant glycoform structures on the immunoglobulins that regulate, e.g., promote a specific effector function.

### BACKGROUND OF THE INVENTION

Glycoproteins mediate many essential functions in humans and other mammals, including catalysis, signaling, cell-cell communication, and molecular recognition and association. Glycoproteins make up the majority of non-cytosolic proteins in eukaryotic organisms (Lis and Sharon, 1993, Eur. J. Biochem. 218:1-27). Many glycoproteins have been exploited for therapeutic purposes, and during the last two decades, recombinant versions of naturally-occurring glycoproteins have been a major part of the biotechnology industry. Examples of recombinant glycosylated proteins used as therapeutics include eiythropoietin (EPO), therapeutic monoclonal antibodies (mAbs), tissue plasminogen activator (tPA), interferon-β (IFN-β), granulocyte-macrophage colony stimulating factor (GM-CSF), and human chorionic gonadotrophin (hCH) (Cumming et al., 1991, Glycobiology 1:115-130). Variations in glycosylation patterns of recombinantly produced glycoproteins have recently been the topic of much attention in the scientific community as recombinant proteins produced as potential prophylactics and therapeutics approach the clinic.

Antibodies or immunoglobulins (Ig) are glycoproteins that play a central role in the humoral immune response. Antibodies may be viewed as adaptor molecules that provide a link between humoral and cellular defense mechanisms. Antigen-specific recognition by antibodies results in the formation of immune complexes that may activate multiple effector mechanisms, resulting in the removal and destruction of the complex. Within the general class of immunoglobulins, five classes of antibodies—IgM, IgD, IgG, IgA, and IgE—can be distinguished biochemically as well as functionally, while more subtle differences confined to the variable region account for the specificity of antigen binding. Amongst these five classes of Igs, there are only two types of light chain, which are termed lambda (λ) and kappa (κ). No functional difference has been found between antibodies having λ or κ chains, and the ratio of the two types of light chains varies from species to species. There are five heavy chain classes or isotypes, and these determine the functional activity of an antibody molecule. The five functional classes of immunoglobulin are: immunoglobulin M (IgM), immunoglobulin D (IgD), immunoglobulin G (IgG), immunoglobulin A (IgA) and immunoglobulin E (IgE). Each isotype has a particular function in immune responses and their distinctive functional properties are conferred by the carboxy-terminal part of the heavy chain, where it is not associated with the light chain. IgG is the most abundant immunoglobulin isotype in blood plasma, (See for example, Immunobiology, Janeway et al, 6^{th} Edition, 2004, Garland Publishing, New York).

The immunoglobulin G (IgG) molecule comprises a Fab (fragment antigen binding) domain with constant and variable regions and an Fc (fragment crystallized) domain. The CH2 domain of each heavy chain contains a single site for N-linked glycosylation at an asparagine residue linking an N-glycan to the Ig molecule, usually at residue Asn-297 (Kabat et al., Sequences of proteins of immunological interest, Fifth Ed., U.S. Department of Health and Human Services, NIH Publication No. 91-3242).

Analyses of the structural and functional aspects of the N-linked oligosaccharides are of biological interest for three main reasons: (1) the glycosylation of the CH2 domain has been conserved throughout evolution, suggesting an important role for the oligosaccharides; (2) the immunoglobulin molecule serves as a model system for the analysis of oligosaccharide heterogeneity (Rademacher and Dwek, 1984; Rademacher et al., 1982); and (3) antibodies comprise dimeric associations of two heavy chains which place two oligosaccharide units in direct contact with each other, so that the immunoglobulin molecule involves both specific protein-carbohydrate and carbohydrate-carbohydrate interactions.

It has been shown that different glycosylation patterns of Igs are associated with different biological properties (Jefferis and Lund, 1997, Antibody Eng. Chem. Immunol., 65: 111-128; Wright and Morrison, 1997, Trends Biotechnol., 15: 26-32). However, only a few specific glycoforms are known to confer desired biological functions. For example, an immunoglobulin composition having decreased fucosylation on N-linked glycans is reported to have enhanced binding to human FcγRIII and therefore enhanced antibody-dependent cellular cytotoxicity (ADCC) (Shields et al., 2002, J. Biol Chem, 277: 26733-26740; Shinkawa et al., 2003, J. Biol. Chem. 278: 3466-3473). And, compositions of fucosylated G2 (Gal₂GlcNAc₂ Man₃GlcNAc₂) IgG made in CHO cells reportedly increase complement-dependent cytotoxicity (CDC) activity to a greater extent than compositions of heterogenous antibodies (Raju, 2004, U.S. Pat. Appl. No. 2004/0136986). It has also been suggested that an optimal antibody against tumors would be one that bound preferentially to activate Fc receptors (FcγRI, FcγRIIa, FcγRIII) and minimally to the inhibitory FcγRIIb receptor (Clynes et al., 2000, Nature, 6:443-446). Therefore, the ability to enrich for specific glycoforms on Ig glycoproteins is highly desirable.

In general, the glycosylation structures (oligosaccharides) on glycoprotein will vary depending upon the expression host and culturing conditions. Therapeutic proteins produced in non-human host cells are likely to contain non-human glycosylation which may elicit an immunogenic response in humans—e.g. hypermannosylation in yeast (Ballou, 1990, Methods Enzymol. 185:440470); α(1,3)-fucose and β(1,2)-xylose in plants, (Cabanes-Macheteau et al., 1999, Glycobiology, 9: 365-372); N-glycolylneuraminic acid in Chinese hamster ovary cells (Noguchi et al., 1995. J. Biochem. 117: 5-62) and Galα-1,3Gal glycosylation in mice (Borrebaeck et al., 1993, Immun. Today, 14: 477-479). Furthermore, galactosylation can vary with cell culture conditions, which may render some immunoglobulin compositions immunogenic depending on their specific galactose pattern (Patel et al., 1992. Biochem J. 285: 839-845). The oligosaccharide structures of glycoproteins produced by non-human mammalian cells tend to be more closely related to those of human glycoproteins. Thus, most commercial immunoglobulins are produced in mammalian cells. However, mammalian cells have several important disadvantages as host cells for protein production. Besides being costly, processes for expressing proteins in mammalian cells produce heterogeneous populations of glycoforms, have low volumetric titers, and require both ongoing viral containment and significant time to generate stable cell lines.

It is understood that different glycoforms can profoundly affect the properties of a therapeutic, including pharmacokinetics, pharmacodynamics, receptor-interaction and tissue-specific targeting (Graddis et al., 2002, Curr Pharm Biotechnol. 3: 285-297). In particular, for antibodies, the oligosaccharide structure can affect properties relevant to protease resistance, the serum half-life of the antibody mediated by the FcRn receptor, binding to the complement complex Cl, which induces complement-dependent cytoxicity (CDC), and binding to FcγR receptors, which are responsible for modulating the antibody-dependent cell-mediated cytoxicity (ADCC) pathway, phagocytosis and antibody feedback. (Nose and Wigzell, 1983; Leatherbarrow and Dwek, 1983; Leatherbarrow et al., 1985; Walker et al., 1989; Carter et al., 1992, Proc. Natl. Acad. Sci. USA, 89: 4285-4289).

Because different glycoforms are associated with different biological properties, the ability to enrich for one or more specific glycoforms can be used to elucidate the relationship between a specific glycoform and a specific biological function. After a desired biological function is associated with a specific glycoform pattern, a glycoprotein composition enriched for the advantageous glycoform structures can be produced. Thus, the ability to produce glycoprotein compositions that are enriched for particular glycoforms is highly desirable.

### SUMMARY OF THE INVENTION

The present invention provides a composition comprising a plurality of immunoglobulins each immunoglobulin comprising at least one N-glycan attached thereto wherein the composition thereby comprises a plurality of N-glycans in which the predominant N-glycan consists essentially of Man₅GlcNAc₂ In preferred embodiments, greater than 50 mole percent of said plurality of N-glycans consists essentially of Man₅GlcNAc₂. More preferably, greater than 75 mole percent of said plurality of N-glycans consists essentially of Man₅GlcNAc₂. Most preferably, greater than 90 percent of said plurality of N-glycans consists essentially of Man₅GlcNAc₂. In other preferred embodiments, said Man₅GlcNAc₂ N-glycan structure is present at a level that is from about 5 mole percent to about 50 mole percent more than the next most predominant N-glycan structure of said plurality of N-glycans.

The present invention also provides methods for increasing binding to FcγRIIIa and FcγRIIIb receptor and decreasing binding to FcγRIIb receptor by enriching for a specific glycoform (e.g. Man₅GlcNAc₂) on an immunoglobulin. A preferred embodiment provides a method for producing a composition comprising a plurality of immunoglobulins, each immunoglobulin comprising at least one N-glycan attached thereto wherein the composition thereby comprises a plurality of N-glycans in which the predominant N-glycan consists essentially of Man₅GlcNAc₂, said method comprising the step of culturing a host cell that has been engineered or selected to express said immunoglobulin or fragment thereof. Another preferred embodiment provides a method for producing a composition comprising a plurality of immunoglobulins, each immunoglobulin comprising at least one N-glycan attached thereto wherein the composition comprises a plurality of N-glycans in which the predominant N-glycan consists essentially of Man₅GlcNAc₂, said method comprising the step of culturing a lower eukaryotic host cell that has been engineered or selected to express said immunoglobulin or fragment thereof. In other embodiments of the present invention, a host cell comprises an exogenous gene encoding an immunoglobulin or fragment thereof, said host cell is engineered or selected to express said immunoglobulin or fragment thereof, thereby producing a composition comprising a plurality of immunoglobulins, each immunoglobulin comprising at least one N-glycan attached thereto wherein the composition thereby comprises a plurality of N-glycans in which the predominant N-glycan consists essentially of Man₅GlcNAc₂. In still other embodiments of the present invention, a lower eukaryotic host cell comprises an exogenous gene encoding an immunoglobulin or fragment thereof, said host cell is engineered or selected to express said immunoglobulin or fragment thereof, thereby producing a composition comprising a plurality of immunoglobulins, each immunoglobulin comprising at least one N-glycan attached thereto wherein the composition thereby comprises a plurality of N-glycans in which the predominant N-glycan consists essentially of Man₅GlcNAc₂.

In preferred embodiments of the present invention, a composition comprising a plurality of immunoglobulins each immunoglobulin comprising at least one N-glycan attached thereto wherein the composition thereby comprises a plurality of N-glycans in which the predominant N-glycan consists essentially of Man₅GlcNAc₂ wherein said immunoglobulins exhibit decreased binding affinity to FcγRIIb receptor. In other preferred embodiments of the present invention, a composition comprising a plurality of immunoglobulins each immunoglobulin comprising at least one N-glycan attached thereto wherein the composition thereby comprises a plurality of N-glycans in which the predominant N-glycan consists essentially of Man₅GlcNAc₂ wherein said immunoglobulins exhibit increased binding affinity to FcγRIIIa and FcγRIIIb receptor. In still another preferred embodiment of the present invention, a composition comprising a plurality of immunoglobulins each immunoglobulin comprising at least one N-glycan attached thereto wherein the composition thereby comprises a plurality of N-glycans in which the predominant N-glycan consists essentially of Man₅GlcNAc₂ wherein said immunoglobulins exhibit increased antibody-dependent cellular cytoxicity (ADCC).

In one embodiment the composition of the present invention comprises immunoglobulins which are essentially free of fucose. In another embodiment, the composition of the present invention comprises immunoglobulins which lack fucose. The composition of the present invention also comprises a pharmaceutical composition and a pharmaceutically acceptable carrier. The composition of the present invention also comprises a pharmaceutical composition of immunoglobulins which have been purified and incorporated into a diagnostic kit.

Accordingly, the present invention provides materials and methods for production of compositions of glycoproteins having predetermined glycosylation structures, in particular, immunoglobulin or antibody molecules having N-glycans consisting essentially of Man₅GlcNAc₂.

### BRIEF DESCRIPTION OF THE DRAWINGS

FIG. 1. Schematic representation of IgG having Man₅GlcNAc₂ glycan structure.

FIG. 2. Coomassie blue stained SDS-PAGE gel of JC-IgG expressed in YJN531-1 (as described in Example 2) and purified from the culture medium (as described in Example 3) over a Protein A column (lane 1) and a phenyl sepharose column (lane 2). (3.0 μg protein/lane.)

FIG. 3. Coomassie blue stained SDS-PAGE gel of DX-IgG expressed in YGLY14 (as described Example 2) and purified from the culture medium (as described in Example 3). Lane 1 shows 2.5 μg of DX-IgG from a phenyl sepharose column.

FIG. 4A. MALDI-TOF spectra of JC-IgG expressed in YJN531-1, treated with α-1,2 mannosidase showing predominantly Man₅GlcNAc₂ N-glycans. FIG. 4B. MALDI-TOF spectrum of DX-IgG expressed in YGLY14, treated with α-1,2 mannosidase showing predominantly Man₅GlcNAc₂ N-glycans.

FIG. 5A. ELISA binding assay of FcγRIIIb with JC-IgG and Rtuximab®. B. ELISA binding assay of FcγRIIIB with DX-IgG and Rituximab®. (M5=Man₅GlcNAc₂ N-glycan).

FIG. 6. ELISA binding assay of FcγRIIIa-158F with JC-IgG and Rituximab®. (M5=Man₅GlcNAc₂ N-glycan).

FIG. 7A. ELISA binding assay of FcγRIIb with JC-IgG and Rituximab®. FIG. 7B. ELISA binding assay of FcγRIIb with DX-IgG and Rituximab®. (M5=Man₅GlcNAc₂ N-glycan).

### BRIEF DESCRIPTION OF THE SEQUENCES

SEQ ID NO: 1 encodes the nucleotide sequence of the murine variable and human constant regions of DX-IgG1 light chain.
SEQ ID NO: 2 encodes the nucleotide sequence of the murine variable and human constant regions of DX-IgG1 heavy chain.
SEQ ID NO: 3 encodes the nucleotide sequence of the human constant region of an IgG1 light chain.
SEQ ID NO: 4 encodes the nucleotide sequence of the human constant region of an IgG1 heavy chain.
SEQ ID NO: 5 to 19 encode 15 overlapping oligonucleotides used to synthesize by polymerase chain reaction (PCR) the murine light chain variable region of DX-IgG 1.
SEQ ID NO: 20 to 23 encode four oligonucleotide primers used to ligate the DX-IgG1 murine light chain variable region to a human light chain constant region.
SEQ ID NO: 24 to 40 encode 17 overlapping oligonucleotides used to synthesize by PCR the murine heavy chain variable region of DX-IgG1.
SEQ ID NO: 41 to 44 encode four oligonucleotide primers used to ligate the DX-IgG1 murine heavy chain variable region to a human heavy chain constant region.
SEQ ID NO: 45 encodes the nucleotide sequence encoding the Kar2 (Bip) signal sequence with an N-terminal EcoRI site.
SEQ ID NO: 46 to 49 encode four oligonucleotide primers used to ligate the Kar2 signal sequence to the light and heavy chains of DX-IgG1.
SEQ ID NO: 50 encodes the nucleotide sequence corresponding to the murine IgG1 variable region of the JC-IgG1 light chain (GenBank #AF013576).
SEQ ID NO: 51 encodes the nucleotide sequence corresponding to the murine IgG1 variable region of the JC-IgG1 heavy chain (GenBank #AF013577).
SEQ ID NO: 52 to 63 encode 12 overlapping oligonucleotide sequences used to PCR-synthesize the murine light chain variable region of JC-IgG1.
SEQ ID NO: 64 to 75 encode 12 overlapping oligonucleotides used to PCR-synthesize the murine heavy chain Fab fragment of JC-IgG1.
SEQ ID NO: 76 to 87 encode 12 overlapping oligonucleotides used to synthesize by PCR the murine heavy chain Fc fragment of JC-IgG1.
SEQ ID NO: 88 encodes a 3′ Kpn1 primer corresponding to the 3′ end of the Fc fragment.
SEQ ID NO: 89 encodes the nucleotide sequence for human serum albumin (HSA).
SEQ ID NO: 90 encodes the nucleotide sequence for thrombin cleavage used in the present invention.

### DETAILED DESCRIPTION OF THE INVENTION

Unless otherwise defined herein, scientific and technical terms and phrases used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of biochemistry, enzymology, molecular and cellular biology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999); Immunobiology, Janeway et al, 6^{th} Edition, 2004, Garland Publishing, New York) All publications, patents and other references mentioned herein are hereby incorporated by reference in their entireties.

The following terms, unless otherwise indicated, shall be understood to have the following meanings:

As used herein, the terms “N-glycan”, “glycan” and “glycoform” are used interchangeably and refer to an N-linked oligosaccharide, e.g., one that is or was attached by an N-acetylglucosamine residue linked to the amide nitrogen of an asparagine residue in a protein. The predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)). The processing of the sugar groups occurs cotranslationally in the lumen of the ER and continues in the Golgi apparatus for N-linked glycoproteins.

N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). N-glycans differ with respect to the number of branches (antennae) comprising peripheral sugars (e.g., GlcNAc, galactose, fucose and sialic acid) that are added to the Man₃GlcNAc₂ (“Man3”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms.”

Abbreviations used herein are of common usage in the art, see, e.g., abbreviations of sugars, above. Other common abbreviations include “PNGase”, or “glycanase” or “glucosidase” which all refer to peptide N-glycosidase F (EC 3.2.2.18).

An “isolated” or “substantially pure” nucleic acid or polynucleotide (e.g., an RNA, DNA or a mixed polymer) is one which is substantially separated from other cellular components that naturally accompany the native polynucleotide in its natural host cell, e.g., ribosomes, polymerases and genomic sequences with which it is naturally associated. The term embraces a nucleic acid or polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, or (4) does not occur in nature. The term “isolated” or “substantially pure” also can be used in reference to recombinant or cloned DNA isolates, chemically synthesized polynucleotide analogs, or polynucleotide analogs that are biologically synthesized by heterologous systems.

However, “isolated” does not necessarily require that the nucleic acid or polynucleotide so described has itself been physically removed from its native environment. For instance, an endogenous nucleic acid sequence in the genome of an organism is deemed “isolated” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “isolated” because it is separated from at least some of the sequences that naturally flank it.

A nucleic acid is also considered “isolated” if it contains any modifications that do not naturally occur to the corresponding nucleic acid in a genome. For instance, an endogenous coding sequence is considered “isolated” if it contains an insertion, deletion or a point mutation introduced artificially, e.g., by human intervention. An “isolated nucleic acid” also includes a nucleic acid integrated into a host cell chromosome at a heterologous site and a nucleic acid construct present as an episome. Moreover, an “isolated nucleic acid” can be substantially free of other cellular material, or substantially free of culture medium when produced by recombinant techniques, or substantially free of chemical precursors or other chemicals when chemically synthesized.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence. The term “degenerate oligonucleotide” or “degenerate primer” is used to signify an oligonucleotide capable of hybridizing with target nucleic acid sequences that are not necessarily identical in sequence but that are homologous to one another within one or more particular segments.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990) (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 50%, more preferably 60% of the nucleotide bases, usually at least about 70%, more usually at least about 80%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51, hereby incorporated by reference. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique, 1:11-15 (1989) and Caldwell and Joyce, PCR Methods Applic. 2:28-33 (1992)); and “oligonucleotide-directed mutagenesis” (a process which enables the generation of site-specific mutations in any cloned DNA segment of interest; see, e.g., Reidhaar-Olson and Sauer, Science 241:53-57 (1988)).

The term “vector” as used herein is intended to refer to a nucleic acid molecule capable of transporting another nucleic acid to which it has been linked. One type of vector is a “plasmid”, which refers to a circular double stranded DNA loop into which additional DNA segments may be ligated. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply, “expression vectors”).

As used herein, the term “sequence of interest” or “gene of interest” refers to a nucleic acid sequence, typically encoding a protein, that is not normally produced in the host cell. The methods disclosed herein allow one or more sequences of interest or genes of interest to be stably integrated into a host cell genome. Non-limiting examples of sequences of interest include sequences encoding one or more polypeptides having an enzymatic activity, e.g., an enzyme which affects N-glycan synthesis in a host such as mannosyltransferases, N-acetylglucosaminyltransferases, UDP-N-acetylglucosamine transporters, galactosyltransferases, UDP-N-acetylgalactosyltransferase, sialyltransferases and fucosyltransferases.

The term “marker sequence” or “marker gene” refers to a nucleic acid sequence capable of expressing an activity that allows either positive or negative selection for the presence or absence of the sequence within a host cell. For example, the P. pastoris URA5 gene is a marker gene because its presence can be selected for by the ability of cells containing the gene to grow in the absence of uracil. Its presence can also be selected against by the inability of cells containing the gene to grow in the presence of 5-FOA. Marker sequences or genes do not necessarily need to display both positive and negative selectability. Non-limiting examples of marker sequences or genes from P. pastoris include ADE1, ARG4, HIS4 and URA3. For antibiotic resistance marker genes, kanamycin, neomycin, geneticin (or G418), paromomycin and hygromycin resistance genes are commonly used to allow for growth in the presence of these antibiotics.

“Operatively linked” expression control sequences refers to a linkage in which the expression control sequence is contiguous with the gene of interest to control the gene of interest, as well as expression control sequences that act in trans or at a distance to control the gene of interest.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “eukaryotic” refers to a nucleated cell or organism, and includes insect cells, plant cells, mammalian cells, animal cells and lower eukaryotic cells.

The term “lower eukaryotic cells” includes yeast, fungi, collar-flagellates, microsporidia, alveolates (e.g., dinoflagellates), stramenopiles (e.g, brown algae, protozoa), rhodophyta (e.g., red algae), plants (e.g., green algae, plant cells, moss) and other protists. Yeast and fungi include, but are not limited to: Pichia sp., such as Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis and Pichia methanolica; Saccharomyces sp., such as Saccharomyces cerevisiae; Hansenula polymorpha, Kluyveromyces sp., such as Kluyveromyces lactis; Candida albicans, Aspergillus nidulans, Aspergillus niger, Aspergillus oryzae, Trichoderma reesei, Chrysosporium lucknowense, Fusarium sp., such as Fusarium gram ineum, Fusarium venenatum; Physcomitrella patens and Neurospora crassa.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” as used herein refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A “modified derivative” refers to polypeptides or fragments thereof that are substantially homologous in primary structural sequence but which include, e.g., in vivo or in vitro chemical and biochemical modifications or which incorporate amino acids that are not found in the native polypeptide. Such modifications include, for example, acetylation, carboxylation, phosphorylation, glycosylation, ubiquitination, labeling, e.g., with radionuclides, and various enzymatic modifications, as will be readily appreciated by those skilled in the art. A variety of methods for labeling polypeptides and of substituents or labels useful for such purposes are well known in the art, and include radioactive isotopes such as ¹²⁵I, ³²P, ³⁵S, and ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., Current Protocols in Molecular Biology, Greene Publishing Associates (1992, and Supplements to 2002) (hereby incorporated by reference).

The term “fusion protein” refers to a polypeptide comprising a polypeptide or fragment coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusions that include the entirety of the proteins of the present invention have particular utility. The heterologous polypeptide included within the fusion protein of the present invention is at least 6 amino acids in length, often at least 8 amino acids in length, and usefully at least 15, 20, and 25 amino acids in length. Fusions that include larger polypeptides, such as an immunoglobulin Fc fragment, or an immunoglobulin Fab fragment or even entire proteins, such as the green fluorescent protein (“GFP”) chromophore-containing proteins or a full length immunoglobulin having particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

As used herein, the terms “antibody”, “immunoglobulin”, “Ig” and “Ig molecule” are used interchangeably. Each antibody molecule has a unique structure that allows it to bind its specific antigen, but all antibodies/immunoglobulins have the same overall structure as described herein. The basic antibody structural unit is known to comprise a tetramer of subunits. Each tetramer has two identical pairs of polypeptide chains, each pair having one “light” chain (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Light chains are classified as either kappa or lambda. Heavy chains are classified as gamma, mu, alpha, delta, or epsilon, and define the antibody's isotype as IgG, IgM, IgA, IgD and IgE, respectively. The light and heavy chains are subdivided into variable regions and constant regions (See generally, Fundamental Immunology (Paul, W., ed., 2nd ed. Raven Press, N.Y., 1989), Ch. 7 (incorporated by reference in its entirety for all purposes). The variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. The terms include naturally occurring forms, as well as fragments and derivatives. Included within the scope of the term are classes of Igs, namely, IgG, IgA, IgE, IgM, and IgD. Also included within the scope of the terms are the subtypes of IgGs, namely, IgG1, IgG2, IgG3 and IgG4. The term is used in the broadest sense and includes single monoclonal antibodies (including agonist and antagonist antibodies) as well as antibody compositions which will bind to multiple epitopes or antigens. The terms specifically cover monoclonal antibodies (including full length monoclonal antibodies), polyclonal antibodies, multispecific antibodies (e.g., bispecific antibodies), and antibody fragments so long as they contain or are modified to contain at least the portion of the C_{H}2 domain of the heavy chain immunoglobulin constant region which comprises an N-linked glycosylation site of the C_{H}2 domain, or a variant thereof. Included within the terms are molecules comprising the Fc region, such as immunoadhesins (U.S. Pat. Appl. No. 2004/0136986), Fc fusions and antibody-like molecules. Alternatively, these terms can refer to an antibody fragment of at least the Fab region that at least contains an N-linked glycosylation site.

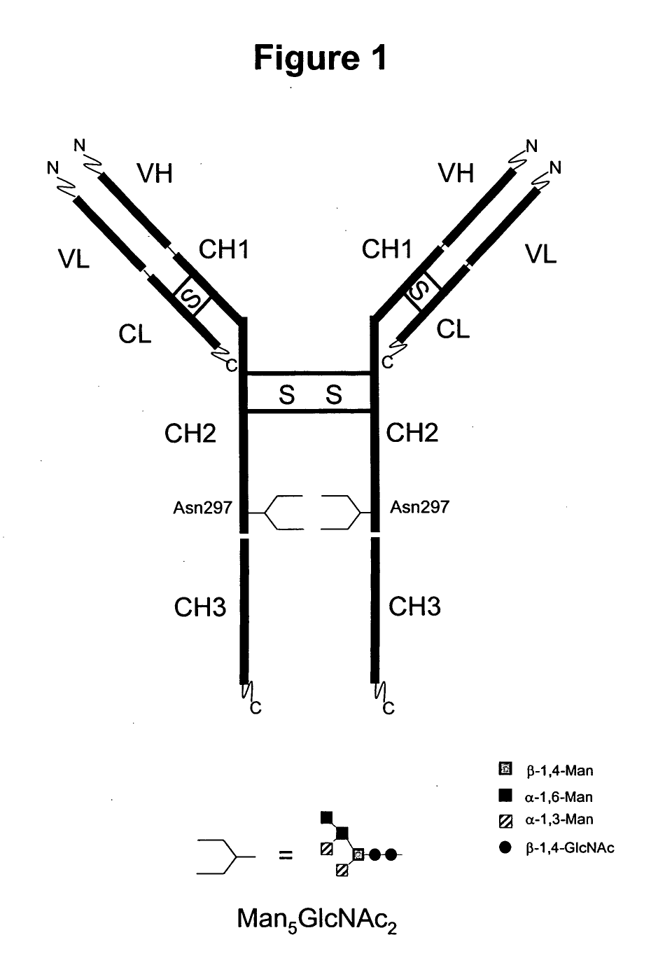

The term “Fc” fragment refers to the ‘fragment crystallized’ C-terminal region of the antibody containing the C_{H}2 and C_{H}3 domains (FIG. 1). The term “Fab” fragment refers to the ‘fragment antigen binding’ region of the antibody containing the VH, CH1, VL and CL domains (FIG. 1).

The term “monoclonal antibody” (mAb) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each mAb is directed against a single determinant on the antigen. In addition to their specificity, monoclonal antibodies are advantageous in that they can be synthesized by hybridoma culture, uncontaminated by other immunoglobulins. The term “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., (1975) Nature, 256:495, or may be made by recombinant DNA methods (see, e.g., U.S. Pat. No. 4,816,567 to Cabilly et al.).

The monoclonal antibodies herein include hybrid and recombinant antibodies produced by splicing a variable (including hypervariable) domain of an antibody with a constant domain (e.g. “humanized” antibodies), or a light chain with a heavy chain, or a chain from one species with a chain from another species, or fusions with heterologous proteins, regardless of species of origin or immunoglobulin class or subclass designation, (See, e.g., U.S. Pat. No. 4,816,567 to Cabilly et al.; Mage and Lamoyi, in Monoclonal Antibody Production Techniques and Applications, pp. 79-97 (Marcel Dekker, Inc., New York, 1987).) The monoclonal antibodies herein specifically include “chimeric” antibodies (immunoglobulins) in which a portion of the heavy and/or light chain is identical with or homologous to corresponding sequences in antibodies derived from a first species or belonging to a particular antibody class or subclass, while the remainder of the chain(s) is identical with or homologous to corresponding sequences in antibodies derived from a different species or belonging to a different antibody class or subclass, as well as fragments of such antibodies, so long as they contain or are modified to contain at least one C_{H}2. “Humanized” forms of non-human (e.g., murine) antibodies are specific chimeric immunoglobulins, immunoglobulin chains or fragments thereof (such as Fv, Fab, Fab′, F(ab′)₂, or other antigen-binding subsequences of antibodies) which contain sequences derived from human immunoglobulins. An Fv fragment of an antibody is the smallest unit of the antibody that retains the binding characteristics and specificity of the whole molecule. The Fv fragment is a noncovalently associated heterodimer of the variable domains of the antibody heavy chain and light chain. The F(ab)′2 fragment is a fragment containing both arms of Fab fragments linked by the disulfide bridges.

The most common forms of humanized antibodies are human immunoglobulins (recipient antibody) in which residues from a complementary-determining region (CDR) of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Furthermore, humanized antibodies can comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the CDR regions correspond to those of a non-human immunoglobulin and all or substantially all of the CDR regions are those of a human immunoglobulin consensus sequence. The humanized antibody optimally also will comprise at least a portion of an immunoglobulin constant region (Fc), typically that of a human immunoglobulin. For further details see Jones et al., 1986, Nature 321:522-524; Reichmann et al., 1988, Nature 332:323-327, and Presta, 1992, Curr. Op. Struct. Biol. 2:593-596.

“Fragments” within the scope of the terms antibody or immunoglobulin include those produced by digestion with various proteases, those produced by chemical cleavage and/or chemical dissociation and those produced recombinantly, so long as the fragment remains capable of specific binding to a target molecule. Among such fragments are Fc, Fab, Fab′, Fv, F(ab′)₂, and single chain Fv (scFv) fragments.

Targets of interest for antibodies of the invention include growth factor receptors (e.g., FGFR, PDGFR, EGFR, NGFR, and VEGF) and their ligands. Other targets are G protein receptors and include substance K receptor, the angiotensin receptor, the α- and β-adrenergic receptors, the serotonin receptors, and PAF receptor. See, e.g., Gilman, Ann. Rev. Biochem. 56:625-649 (1987). Other targets include ion channels (e.g., calcium, sodium, potassium channels), muscarinic receptors, acetylcholine receptors, GABA receptors, glutamate receptors, and dopamine receptors (see Harpold, U.S. Pat. No. 5,401,629 and U.S. Pat. No. 5,436,128). Other targets are adhesion proteins such as integrins, selectins, and immunoglobulin superfamily members (see Springer, Nature 346:425-433 (1990). Osborn, Cell 62:3 (1990); Hynes, Cell 69:11 (1992)). Other targets are cytokines, such as interleukins IL-1 through IL-13, tumor necrosis factors α & β, interferons α, β and γ, tumor growth factor Beta (TGF-β), colony stimulating factor (CSF) and granulocyte monocyte colony stimulating factor (GMCSF). See Human Cytokines: Handbook for Basic & Clinical Research (Aggrawal et al. eds., Blackwell Scientific, Boston, Mass. 1991). Other targets are hormones, enzymes, and intracellular and intercellular messengers, such as, adenyl cyclase, guanyl cyclase, and phospholipase C. Other targets of interest are leukocyte antigens, such as CD20, and CD33. Drugs may also be targets of interest. Target molecules can be human, mammalian or bacterial. Other targets are antigens, such as proteins, glycoproteins and carbohydrates from microbial pathogens, both viral and bacterial, and tumors. Still other targets are described in U.S. Pat. No. 4,366,241.

Immune Fc receptors discussed herein, may include: FcγRI, FcγRIIa, FcγRIIb, FcγRIIIa, FcγRIIIb and FcRn (neonatal receptor). The term FcγRI can refer to any FcγRI subtype unless specified otherwise. The term FcγRII can refer to any FcγRII receptor unless specified otherwise. The term FcγRIII refers to any FcγRIII subtype unless specified otherwise.

“Derivatives” within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Intracellular Antibodies: Research and Disease Applications, (Marasco, ed., Springer-Verlag New York, Inc., 1998).

The term “non-peptide analog” refers to a compound with properties that are analogous to those of a reference polypeptide. A non-peptide compound may also be termed a “peptide mimetic” or a “peptidomimetic”. See, e.g., Jones, Amino Acid and Peptide Synthesis, Oxford University Press (1992); Jung, Combinatorial Peptide and Nonpeptide Libraries. A Handbook, John Wiley (1997); Bodanszky et al., Peptide Chemistry—A Practical Textbook, Springer Verlag (1993); Synthetic Peptides: A Users Guide, (Grant, ed., W. H. Freeman and Co., 1992); Evans et al., J. Med. Chem. 30:1229 (1987); Fauchere, J. Adv. Drug Res. 15:29 (1986); Veber and Freidinger, Trends Neurosci., 8:392-396 (1985); and references sited in each of the above, which are incorporated herein by reference. Such compounds are often developed with the aid of computerized molecular modeling. Peptide mimetics that are structurally similar to useful peptides of the invention may be used to produce an equivalent effect and are therefore envisioned to be part of the invention.

Amino acid substitutions can include those which: (1) reduce susceptibility to proteolysis, (2) reduce susceptibility to oxidation, (3) alter binding affinity for forming protein complexes, (4) alter binding affinity or enzymatic activity, and (5) confer or modify other physicochemical or functional properties of such analogs.

As used herein, the twenty conventional amino acids and their abbreviations follow conventional usage. See Immunology—A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) In a preferred embodiment, a homologous protein is one that exhibits at least 65% sequence homology to the wild type protein, more preferred is at least 70% sequence homology. Even more preferred are homologous proteins that exhibit at least 75%, 80%, 85% or 90% sequence homology to the wild type protein. In a yet more preferred embodiment, a homologous protein exhibits at least 95%, 98%, 99% or 99.9% sequence identity. As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A preferred algorithm when comparing a particular polypepitde sequence to a database containing a large number of sequences from different organisms is the computer program BLAST (Altschul et al., J. Mol. Biol. 215:403410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (herein incorporated by reference). For example, percent sequence identity between amino acid sequences can be determined using FASTA with its default parameters (a word size of 2 and the PAM250 scoring matrix), as provided in GCG Version 6.1, herein incorporated by reference.

“Specific binding” refers to the ability of two molecules to bind to each other in preference to binding to other molecules in the environment. Typically, “specific binding” discriminates over adventitious binding in a reaction by at least two-fold, more typically by at least 10-fold, often at least 100-fold. Typically, the affinity or avidity of a specific binding reaction, as quantified by a dissociation constant, is about 10⁻⁷ M or stronger (e.g., about 10⁻⁸ M, 10⁻⁹ M or even stronger).

The term “region” as used herein refers to a physically contiguous portion of the primary structure of a biomolecule. In the case of proteins, a region is defined by a contiguous portion of the amino acid sequence of that protein.

The term “domain” as used herein refers to a structure of a biomolecule that contributes to a known or suspected function of the biomolecule. Domains may be co-extensive with regions or portions thereof; domains may also include distinct, non-contiguous regions of a biomolecule.

As used herein, the term “molecule” means any compound, including, but not limited to, a small molecule, peptide, protein, glycoprotein, sugar, nucleotide, nucleic acid, lipid, etc., and such a compound can be natural or synthetic.

As used herein, the term “comprise” or variations such as “comprises” or “comprising”, will be understood to imply the inclusion of a stated integer or group of integers but not the exclusion of any other integer or group of integers.

As used herein, the term “consisting essentially of” will be understood to imply the inclusion of a stated integer or group of integers; while excluding modifications or other integers which would materially affect or alter the stated integer. With respect to species of N-glycans, the term “consisting essentially of” a stated N-glycan will be understood to include the N-glycan whether or not that N-glycan is fucosylated at the N-acetylglucosamine (GlcNAc) which is directly linked to the asparagine residue of the glycoprotein.

As used herein, the term “predominantly” or variations such as “the predominant” or “which is predominant” will be understood to mean the glycan species that has the highest mole percent (%) of total N-glycans after the glycoprotein has been treated with PNGase and released glycans analyzed by mass spectroscopy, for example, MALDI-TOF MS. In other words, the phrase “predominantly” is defined as an individual entity, such as a specific glycoform, is present in greater mole percent than any other individual entity. For example, if a composition consists of species A in 40 mole percent, species B in 35 mole percent and species C in 25 mole percent, the composition comprises predominantly species A, and species B would be the next most predominant species.

As used herein, the term “essentially free of” a particular sugar residue, such as fucose, or galactose and the like, is used to indicate that the glycoprotein composition is substantially devoid of N-glycans which contain such residues. Expressed in terms of purity, essentially free means that the amount of N-glycan structures containing such sugar residues does not exceed 10%, and preferably is below 5%, more preferably below 1%, most preferably below 0.5%, wherein the percentages are by weight or by mole percent. Thus, substantially all of the N-glycan structures in a glycoprotein composition according to the present invention are free of fucose, or galactose, or both.

As used herein, a glycoprotein composition “lacks” or “is lacking” a particular sugar residue, such as fucose or galactose, when no detectable amount of such sugar residue is present on the N-glycan structures at any time. For example, in preferred embodiments of the present invention, the glycoprotein compositions are produced by lower eukaryotic organisms, as defined above, including yeast [e.g., Pichia sp.; Saccharomyces sp.; Kluyveromyces sp.; Aspergillus sp.], and will “lack fucose,” because the cells of these organisms do not have the enzymes needed to produce fucosylated N-glycan structures. Thus, the term “essentially free of fucose” encompasses the term “lacking fucose.” However, a composition may be “essentially free of fucose” even if the composition at one time contained fucosylated N-glycan structures or contains limited, but detectable amounts of fucosylated N-glycan structures as described above.

As used herein, the phrase “increased binding activity” is used interchangeably with “increased binding affinity” referring to an increase in the binding of the IgG molecule with a receptor—or otherwise noted molecule.

As used herein, the phrase “decreased binding activity” is used interchangeably with “decreased binding affinity” referring to a decrease in the binding of the IgG molecule with a receptor—or otherwise noted molecule.

As used herein, the phrase, “phagocytosis” is defined to be clearance of immunocomplexes. Phagocytosis is an immunological activity of immune cells-including but not limited to, macrophages and neutrophils.

The interaction of antibodies and antibody-antigen complexes with cells of the immune system and the variety of responses, including antibody-dependent cell-mediated cytotoxicity (ADCC) and complement-dependent cytotoxicity (CDC), clearance of immunocomplexes (phagocytosis), antibody production by B cells and IgG serum half-life are defined respectively in the following: Daeron et al., 1997, Annu. Rev. Immunol. 15: 203-234; Ward and Ghetie, 1995, Therapeutic Immunol. 2:77-94; Cox and Greenberg, 2001, Semin. Immunol. 13: 339-345; Heyman, 2003, Immunol. Lett. 88:157-161; and Ravetch, 1997, Curr. Opin. Immunol. 9: 121-125.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

Recombinant Ig-Man₅GlcNAc₂ Molecules

The present invention provides compositions comprising a population of glycosylated Igs having a predominant Man₅GlcNAc₂ N-linked glycoform. The present invention also provides Igs and Ig compositions having a predominant Man₅GlcNAc₂ N-linked glycoform that mediates antibody effector functions, such as receptor binding. Preferably the interaction between an Ig of the present invention and an FcγRIII receptor provides an increase in direct binding activity. And, preferably the interaction between an Ig of the present invention and the FcγRIIb receptor provides a decrease (or lack of) direct binding activity. In another embodiment, an Ig or Ig composition of the present invention exhibits increased binding activity conferred by the enrichment/predominance of a glycoform structure. A salient feature of the present invention is that it provides Igs and Ig compositions having a predominant, specific glycoform that mediates antibody effector functions, such as an increase in ADCC activity or an increase in antibody production by B cells. In another embodiment, an Ig or Ig composition of the present invention exhibits increased ADCC activity or antibody production by B cells conferred by the enrichment/predominance of one glycoform. Furthermore, it will be readily apparent to a skilled artisan that one advantage of producing Ig compositions having a predominant glycoform is that it avoids production of Igs having undesired glycoforms and/or production of heterogeneous mixtures of Igs which may induce undesired effects and/or dilute the concentration of the more effective Ig glycoform(s). It is, therefore, contemplated that a pharmaceutical composition comprising Igs having predominantly Man₅GlcNAc₂ glycoforms will have beneficial features, including but not limited to, decreased binding to FcγRIIb and increased binding to FcγRIIIa and FcγRIIIb, and therefore may well be effective at lower doses, thus having higher efficacy/potency.

In one embodiment, an Ig molecule of the present invention comprises at least one Man₅GlcNAc₂ glycan structure at Asn-297 of a CH² domain of a heavy chain on the Fc region mediating antibody effector function in an Ig molecule. Preferably, the Man₅GlcNAc₂ glycan structure is on each Asn-297 of each C_{H}2 region in a dimerized Ig (FIG. 1). In another embodiment, the present invention provides compositions comprising Igs which are predominantly glycosylated with an N-glycan consisting essentially of Man₅GlcNAc₂ glycan structure at Asn-297 (FIG. 1). Alternatively, one or more carbohydrate moieties found on an Ig molecule may be deleted and/or added to the molecule, thus adding or deleting the number of glycosylation sites on an Ig. Further, the position of the N-linked glycosylation site within the C_{H}2 region of a Ig molecule can be varied by introducing asparagines (Asn) or N-glycosylation sites at varying locations within the molecule. While Asn-297 is the N-glycosylation site typically found in murine and human IgG molecules (Kabat et al., Sequences of Proteins of Immunological Interest, 1991), this site is not the only site that can be envisioned, nor does this site necessarily have to be maintained for function. Using known methods for mutagenesis, the skilled artisan can alter a DNA molecule encoding an Ig of the present invention so that the N-glycosylation site at Asn-297 is deleted, and can further alter the DNA molecule so that one or more N-glycosylation sites are created at other positions within the Ig molecule. It is preferred that N-glycosylation sites are created within the C_{H}2 region of the Ig molecule. However, glycosylation of the Fab region of an Ig has been described in 30% of serum antibodies-commonly found at Asn-75 (Rademacher et al., 1986, Biochem. Soc. Symp., 51: 131-148). Glycosylation in the Fab region of an Ig molecule is an additional site that can be combined in conjunction with N-glycosylation in the Fc region, or alone.

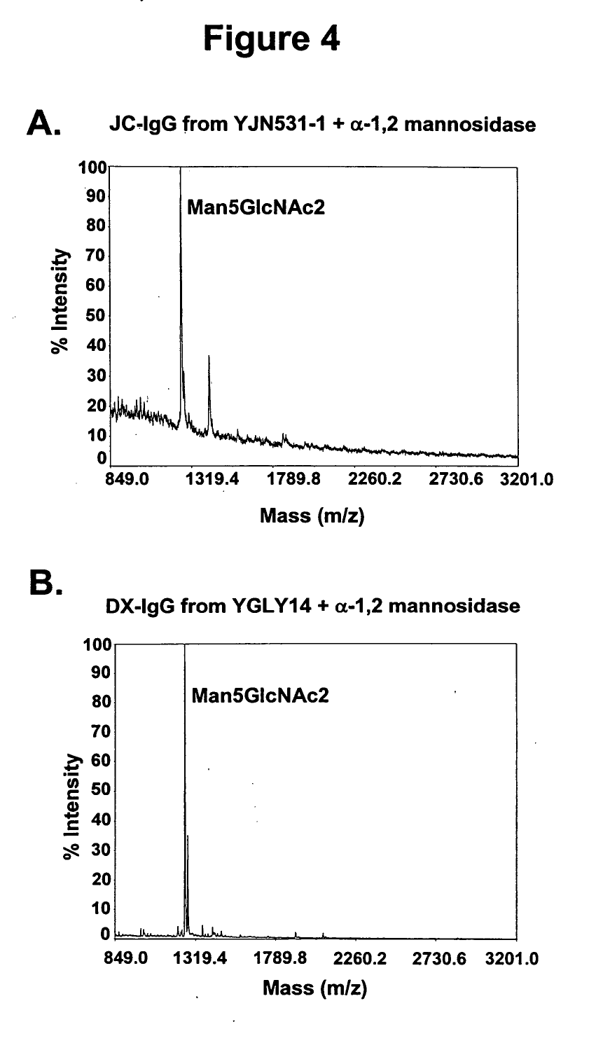

In one embodiment, the present invention provides a recombinant Ig composition having a predominant Man₅GlcNAc₂ N-glycan structure, wherein said Man₅GlcNAc₂ glycan structure is present at a level that is at least about 5 mole percent more than the next predominant glycan structure of the recombinant Ig composition. In a preferred embodiment, the present invention provides a recombinant Ig composition having a predominant Man₅GlcNAc₂ glycan structure, wherein said Man₅GlcNAc₂ glycan structure is present at a level of at least about 10 mole percent to about 25 mole percent more than the next predominant glycan structure of the recombinant Ig composition. In a more preferred embodiment, the present invention provides a recombinant Ig composition having a predominant Man₅GlcNAc₂ glycan structure, wherein said Man₅GlcNAc₂ glycan structure is present at a level that is at least about 25 mole percent to about 50 mole percent more than the next predominant glycan structure of the recombinant Ig composition. In a preferred embodiment, the present invention provides a recombinant Ig composition having a predominant Man₅GlcNAc₂ glycan structure, wherein said Man₅GlcNAc₂ glycan structure is present at a level that is greater than about 50 mole percent more than the next predominant glycan structure of the recombinant Ig composition. In another preferred embodiment, the present invention provides a recombinant Ig composition having a predominant Man₅GlcNAc₂ glycan structure, wherein said Man₅GlcNAc₂ glycan structure is present at a level that is greater than about 75 mole percent more than the next predominant glycan structure of the recombinant Ig composition. In still another embodiment, the present invention provides a recombinant Ig composition having a predominant Man₅GlcNAc₂ glycan structure, wherein said Man₅GlcNAc₂ glycan structure is present at a level that is greater than about 90 mole percent more than the next predominant glycan structure of the recombinant Ig composition. MALDI-TOF analysis of N-glycans of JC-IgG having a predominant Man₅GlcNAc₂ N-glycan (72%) is shown in FIG. 4A. MALDI-TOF analysis of N-glycans of DX-IgG having a predominant Man₅GlcNAc₂ (90%) is shown in FIG. 4B.

Increased Binding of Ig-Man₅GlcNAc₂ to FcγRIII Receptor

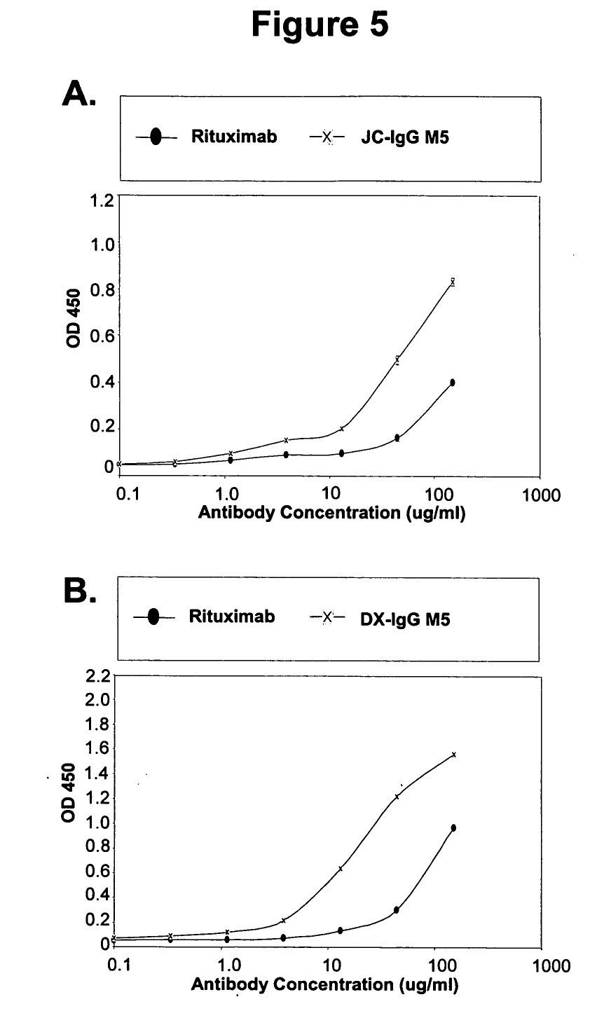

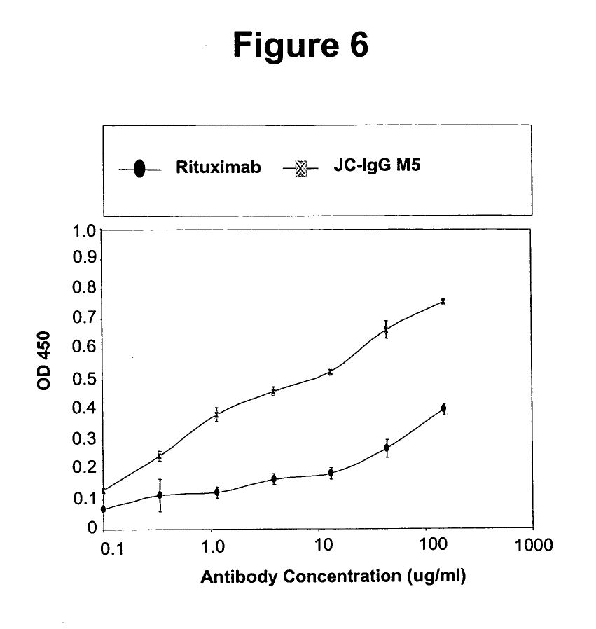

The effector functions of Ig binding to FcγRIIIa and FcγRIIIb, such as activation of ADCC, are mediated by the Fc region of the Ig molecule. Different functions are mediated by the different domains in this region. Accordingly, the present invention provides Ig molecules and compositions in which an Fc region on an Ig molecule has a predominant Man₅GlcNAc₂ N-glycan capable of carrying out an effector function. In one embodiment, the Fc region having a predominant Man₅GlcNAc₂ N-glycan confers an increase in binding to FcγRIIIa (FIG. 6) and FcγRIIIb (FIG. 5) receptors. In another embodiment, an Fc has a predominant Man₅GlcNAc₂ N-glycan. It will be readily apparent to the skilled artisan that molecules comprising the Fc region, such as immunoadhesions (Chamow and Ashkenazi, 1996, Trends Biotechnol. 14: 52-60; Ashkenazi and Chamow, 1997, Curr Opin. Immunol. 9: 195-200), Fc fusions and antibody-like molecules are also encompassed in the present invention.

Binding activity (affinity) of an Ig molecule to an Fc receptor may be determined by an assay. An example of an FcγRIII binding assay with IgG is described in Example 6. One skilled in the art recognizes that this assay can be easily adapted for use in conjunction with assays for any immunoglobulin molecule.

JC-IgG (an Ig made according to the present invention) having predominantly Man₅GlcNAc₂ N-glycans has 5-10 fold increased binding activity to FcγRIIIb compared with Rituximab® as shown in FIG. 5A, and has greater than 10-fold increased binding activity to FcγRIIIa compared with Rituximab® as shown in FIG. 6. DX-IgG (another Ig made according to the present invention) having predominantly Man₅GlcNAc₂ N-glycans also has 5-10 fold increased binding activity to FcγRIIIb compared with Rituximab® as shown in FIG. 5B.

Most interestingly, FcγRIIIa gene dimorphism generates two allotypes: FcγRIIIa-158V and FcγRIIIa-158F (Dall'Ozzo et al., 2004, Cancer Res. 64: 4664-4669). The genotype homozygous for FcγRIIIa-158V is associated with a higher clinical response to Rituximab® (Cartron et al., 2002, Blood, 99: 754-758). However, most of the population carries one FcγRIIIa-158F allele, rendering Rituximab® less effective for most of the population for induction of ADCC through FcγRIIIa binding. However, when a Rituximab®-like anti-CD20 antibody is expressed in a host cell which lacks fucosyltransferase activity, this antibody is equally effective for enhancing ADCC through both FcγRIIIa-158F and FcγRIIIa-158V (Niwa et al., 2004, Clin. Canc Res. 10: 6248-6255). The antibodies of certain preferred embodiments of the present invention are expressed in host cells that do not add fucose to N-glycans (e.g., P. pastoris, a yeast host lacking fucose; see Examples 1 and 2). Therefore, it is contemplated that the antibodies of the present invention that lack fucose and have enhanced binding to FcγRIIIa-158F may be especially useful for treating many patients exhibiting a reduced clinical response to Rituximab®.

Decreased Binding of Ig-Man₅GlcNAc₂ to FcγRIIb Receptor

The effector functions of Ig binding to FcγRIIb, such as increased antibody production by B cells and increased ADCC activity, are mediated by the Fc region of the Ig molecule. Different functions are mediated by the different domains in this region. Accordingly, the present invention provides Ig molecules and compositions in which an Fc region on an Ig molecule has a predominant Man₅GlcNAc₂ N-glycan capable of carrying out an effector function. In one embodiment, an Fc region of an Ig having a predominant Man₅GlcNAc₂ N-glycan confers a decrease in binding to an FcγRIIb receptor. It will be readily apparent to the skilled artisan that molecules comprising an Fc region, such as immunoadhesions (Chamow and Ashkenazi, 1996, Trends Biotechnol. 14: 52-60; Ashkenazi and Chamow, 1997, Curr Opin. Immunol. 9: 195-200), Fc fusions and antibody-like molecules are also encompassed in the present invention.

Binding activity (affinity) of an Ig molecule to an Fc receptor may be determined by an assay. An example of an FcγRIIb binding assay with IgG1 is disclosed in Example 6. One skilled in the art recognizes that this disclosed assay can be easily adapted for use in connection to any immunoglobulin molecule.

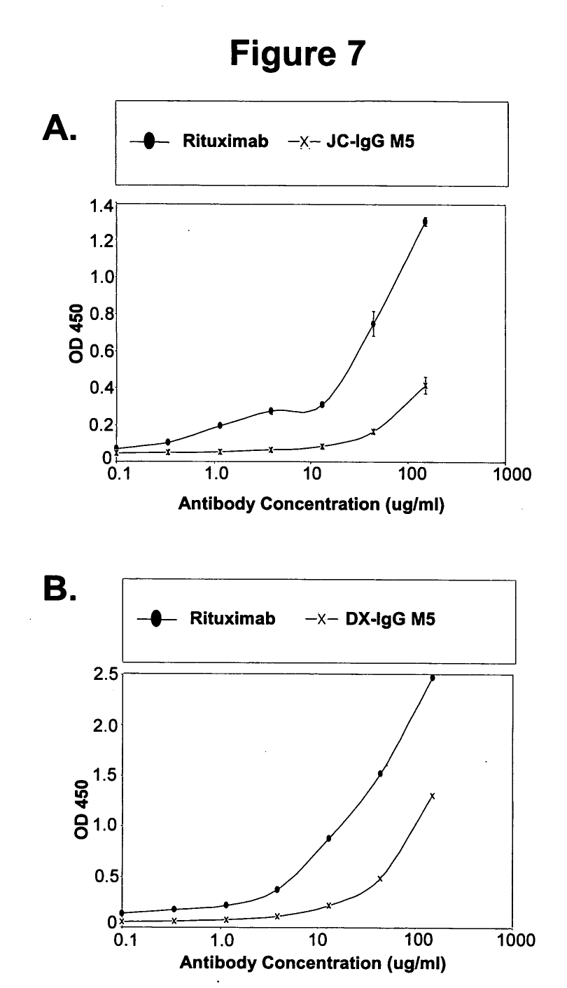

JC-IgG (an Ig of the present invention) having predominant Man₅GlcNAc₂ N-glycans, has 8-fold decreased binding activity to FcγRIIb compared with Rituximab® as shown in FIG. 7A. DX-IgG (another Ig of the present invention) having predominant Man₅GlcNAc₂ N-glycans, also has 8-fold decreased binding activity to FcγRIIb compared with Rituximab® as shown in FIG. 7B.

Increased Antibody-Dependent Cell-Mediated Cytoxicity

In yet another embodiment, the increase in FcγRIIIa or FcγRIIIb binding of an Ig molecule or composition having Man₅GlcNAc₂ as the predominant N-glycan may confer an increase in FcγRIII-mediated ADCC. It is well established that the FcγRIII (CD 16) receptor is responsible for ADCC activity (Daeron et al., 1997, Annu. Rev. Immunol. 15: 203-234). In another embodiment, the decrease in FcγRIIb binding of an Ig molecule or composition having Man₅GlcNAc₂ as the predominant N-glycan confers an increase in ADCC (Clynes et al., 2000, supra). In another embodiment, an Ig molecule or composition of the present invention exhibits increased ADCC activity conferred by the presence of a predominant Man₅GlcNAc₂ glycan.

An example of in vitro assays measuring B-cell depletion and fluorescence release ADCC assays are disclosed in Example 7. One skilled in the art recognizes that these disclosed assays can be easily adapted for use in conjunction with assays for any Ig molecule. Furthermore, an in vivo ADCC assay in an animal model can be adapted for any specific IgG from Borchmann et al., 2003, Blood, 102: 3737-3742, Niwa et al., 2004, Cancer Research, 64: 2127-2133 and Example 7.

Increased Antibody Production by B Cells

Antibody engagement against tumors through the regulatory FcγR pathways has been shown (Clynes et al., 2000, Nature, 6: 443-446). Specifically, it is known when FcγRIIb is co-cross-linked with immunoreceptor tyrosine based activation motifs (ITAM)-containing receptors such as the B cell receptor (BCR), FcγRI, FcγRIII, and FcεRI, it inhibits ITAM-mediated signals (Vivier and Daeron, 1997, Immunol. Today, 18: 286-291). For example, the addition of FcgRII-specific antibodies blocks Fc binding to the FcgRIIB, resulting in augmented B cell proliferation (Wagle et al., 1999, J of Immunol. 162: 2732-2740). Accordingly, in one embodiment, an Ig molecule of the present invention can mediate a decrease in FcγRIIb receptor binding resulting in the activation of B cells which in turn, catalyzes antibody production by plasma cells (Parker, D. C. 1993, Annu. Rev. Immunol. 11: 331-360). An example of an assay measuring antibody production by B cells with IgG1 is described in Example 6. One skilled in the art recognizes that this assay can be easily adapted for use in conjunction with assays for any immunoglobulin molecule.

Other Immunological Activities

Altered surface expression of effector cell molecules on neutrophils has been shown to increase susceptibility to bacterial infections (Ohsaka et al., 1997, Br. J. Haematol. 98: 108-113). It has been further demonstrated that IgG binding to the FcγRIIIa effector cell receptors regulates expression of tumor necrosis factor alpha (TNF-α) (Blom et al., 2004, Arthritis Rheum., 48: 1002-1014). Furthermore, FcγR-induced TNF-α also increases the ability of neutrophils to bind and phagocytize IgG-coated erythrocytes (Capsoni et al., 1991, J. Clin. Lab Immunol. 34: 115-124). It is therefore contemplated that the Ig molecules and compositions of the present invention that show an increase in binding to FcγRIII, may confer an increase in expression of TNF-α.

An increase in FcγRIII receptor activity has been shown to increase the secretion of lysosomal beta-glucuronidase as well as other lysosomal enzymes (Kavai et al., 1982, Adv. Exp Med. Biol. 141: 575-582; Ward and Ghetie, 1995, Therapeutic Immunol., 2: 77-94). Furthermore, an important step after the engagement of immunoreceptors by their ligands is their internalization and delivery to lysosomes (Bonnerot et al., 1998, EMBO J, 17: 4906-4916). It is therefore contemplated that an Ig molecule or composition of the present invention that shows an increase in binding to FcγRIIIa and FcγRIIIb may confer an increase in the secretion of lysosomal enzymes.

Present exclusively on neutrophils, FcγRIIIb plays a predominant role in the assembly of immune complexes, and its aggregation activates phagocytosis, degranulation, and the respiratory burst leading to destruction of opsonized pathogens. Activation of neutrophils leads to secretion of a proteolytically cleaved soluble form of the receptor corresponding to its two extracellular domains. Soluble FcγRIIIb exerts regulatory functions by competitive inhibition of FcγR-dependent effector functions and via binding to the complement receptor CR3, leading to production of inflammatory mediators (Sautes-Fridman et al., 2003, ASHI Quarterly, 148-151).

The present invention thus provides an immunoglobulin molecule comprising an N-glycan consisting essentially of Man₅GlcNAc₂; and provides a composition comprising immunoglobulins and a plurality of N-glycans attached thereto, wherein the predominant N-glycan within said plurality of N-glycans consists essentially of Man₅GlcNAc₂. In either embodiment, the predominance of said Man₅GlcNAc₂ N-glycan on an immunoglobulin preferably confers desired therapeutic effector activity in addition to the improved binding to FcγRIIIa and FcγRIIIb and decreased binding to FcγRIIb, as shown herein.

Immunoglobulin Subclasses

The IgG subclasses have been shown to have different binding affinities for Fc receptors (Huizinga et al., 1989, J. of Immunol., 142: 2359-2364). Each of the IgG subclasses may offer particular advantages in different aspects of the present invention. Thus, in one aspect, the present invention provides an IgG1 composition that comprises Man₅GlcNAc₂ as the predominant N-glycan attached to IgG1 molecules. In another aspect, the present invention comprises an IgG2 composition that comprises Man₅GlcNAc₂ as the predominant N-glycan attached to IgG2 molecules. In yet another aspect, the present invention comprises an IgG3 composition that comprises Man₅GlcNAc₂ as the predominant N-glycan attached to IgG3 molecules. In another aspect, the present invention comprises an IgG4 composition that comprises Man₅GlcNAc₂ as the predominant N-glycan attached to IgG4 molecules.

Alternatively, the present invention can be applied to all of the five major classes of immunoglobulins: IgA, IgD, IgE, IgM and IgG. A preferred immunoglobulin of the present invention is a human IgG and preferably from one of the subtypes IgG1, IgG2, IgG3 or IgG4. More preferably, an immunoglobulin of the present invention is an IgG1 molecule.

Production of Recombinant Immunoglobulin (Ig Molecules Mediating Antibody Effector Function and Activity

In one aspect, the invention provides a method for producing a recombinant Ig molecule having an N-glycan consisting essentially of a Man₅GlcNAc₂ glycan structure at Asn-297 of the C_{H}2 domain, wherein the Ig molecule mediates antibody effector function and activity, and similarly, an immunoglobulin composition wherein the predominant N-glycan attached to the immunoglobulins is Man₅GlcNAc₂. In one embodiment, the heavy and light chains of the Ig are synthesized using overlapping oligonucleotides and are separately cloned into an expression vector (Example 1) for expression in a host cell. In a preferred embodiment, recombinant Ig heavy and light chains are expressed in a host strain which catalyzes predominantly the addition of Man₅GlcNAc₂. In one embodiment, this glycoform structure is more specifically denoted as [Manα1,3 (Manα1,3 Manα1,6 Manα1,6)-Manβ1,4-GlcNAc β1,4-GlcNAc]forming a linkage between the nitrogen of the amino acid Asn-297 of the Fc region on an Ig and the hydroxyl group of N-acetyl-β-D-glucosamine on the Man₅GlcNAc₂ glycan. In yet another embodiment, this predominant glycan can be added to an asparagine at a different site within the Ig molecule (other than Asn-297), or in combination with the N-glycosylation site in the Fab region.

Production of Ig having Predominantly Man₅GlcNAc₂ in Lower Eukaryotes

One aspect of the present invention provides recombinant lower eukaryotic host cells which may be used to produce immunoglobulin or antibody molecules with predominantly the Man₅GlcNAc₂ glycoform, which is an advantage compared with compositions of glycoproteins expressed in mammalian cells which naturally produce said glycoform in low yield.

It is another advantage of the present invention that compositions of glycoproteins are provided with predetermined glycosylation patterns that are readily reproducible. The properties of such compositions are assessed and optimized for desirable properties, while adverse effects may be minimized or avoided altogether.

The present invention also provides methods for producing recombinant host cells that are engineered or selected to express one or more nucleic acids for the production of Ig molecules comprising an N-glycan consisting essentially of Man₅GlcNAc₂ and Ig compositions having predominantly a Man₅GlcNAc₂ glycan structure. In certain preferred embodiments of the present invention, recombinant host cells, preferably recombinant lower eukaryotic host cells, are used to produce said Ig molecules and compositions having predominantly Man₅GlcNAc₂ glycan.

In other preferred embodiments, the invention comprises the glycoproteins obtainable from recombinant host cells or by the methods of the present invention.

The host cells of the invention may be transformed with vectors encoding the desired Ig regions, and with vectors encoding one or more of the glycosylation-related enzymes described herein, and then selected for expression of a recombinant Ig molecule or composition having a predominant Man₅GlcNAc₂ N-glycan. The recombinant host cell of the present invention may be a eukaryotic or prokaryotic host cell, such as an animal, plant, insect, bacterial cell, or the like which has been engineered or selected to produce an Ig composition having predominantly Man₅GlcNAc₂ N-glycan structures.

Preferably, the recombinant host cell of the present invention is a lower eukaryotic host cell which has been genetically engineered as described in the art (WO 02/00879, WO 03/056914, WO 04/074498, WO 04/074499, Choi et al., 2003, PNAS, 100: 5022-5027; Hamilton et al., 2003, Nature, 301: 1244-1246 and Bobrowicz et al., 2004, Glycobiology, 14: 757-766). Specifically, WO 02/00879 discloses a glycoprotein having Man₅GlcNAc₂ N-glycans, and WO 04/074499 discloses glycoproteins (and specific disclosure of immunoglobulins) with predominantly Man₅GlcNAc₂ N-glycans.

In one embodiment, a vector encoding an IgG1, for example an AOX1/pPICZA vector containing JC-IgG (Example 1) is introduced into the yeast P. pastoris YJN531-1 strain which is similar to strain BK64-1 (Choi et al., 2003, supra), except the YJN531-1 strain has had the MNN4b gene disrupted as disclosed in U.S. patent application Ser. No. 11/020,808 and also lacks β-mannosylation by disruption of the AMR2 gene, as described (U.S. patent application Ser. No. 11/118008). This YJN531-1 strain expresses glycoproteins having predominantly Man₈GlcNAc₂, thus, resulting in JC-IgG having predominantly Man₈GlcNAc₂. Treatment of this JC-IgG having predominantly Man₈GlcNAc₂ with α-1,2 mannosidase (Example 3) results in JC-IgG having predominant Man₅GlcNAc₂ N-glycans (FIG. 4A).

In another embodiment, the vector encoding an IgG1 in AOX1/pPICZA containing DX-IgG (Example 1) is introduced in the yeast P. pastoris YGLY14 strain, which is similar to strain BK64-1 (Choi et al., 2003, supra), except the YGLY 14 strain has had the PNO1 and MNN4b genes disrupted as disclosed in U.S. patent application Ser. No. 11/020,808 and also lacks β-mannosylation by disruption of the AMR2 gene, as described (U.S. patent application Ser. No. 11/118,008). This YGLY14 strain expresses glycoproteins having predominantly Man8GlcNAc₂, thus, resulting in DX-IgG having predominantly Man₈GlcNAc₂. Treatment of this DX-IgG having predominantly Man₈GlcNAc₂ with α-1,2 mannosidase (Example 3) results in DX-IgG having predominant Man₅GlcNAc₂ N-glycans (FIG. 4B).

Alternatively, an antibody of the present invention can be expressed using several methods known in the art (Monoclonal Antibody Production Techniques and Applications, pp. 79-97 (Marcel Dekker, Inc., New York, 1987).

Expression of Glycosyltransferases and Stable Genetic Integration in Lower Eukaryotes

Methods for introducing and confirming integration of heterologous genes in a lower eukaryotic host strain (e.g. P. pastoris) using selectable markers such as URA3, URA5, HIS4, SUC2, G418, BLA or SHBLA have been described. Such methods may be adapted to produce an Ig of the present invention when the expression system is produced in a lower eukaryote. Additionally, methods have been described that allow for repeated use of the URA3 marker to eliminate undesirable mannosyltransferase activities. Alani et al., 1987, Genetics, 116: 541-545 and U.S. Pat. No. 6,051,419 describe a selection system based on disrupting the URA3 gene in P. pastoris. Preferably, the PpURA3- or PpURA5-blaster cassettes are used to disrupt the URA3, URA5 or any gene in the uracil biosynthesis pathway, allowing for both positive and negative selection, based on auxotrophy for uracil and resistance to 5-fluoroorotic acid (5FOA) (Boeke, et al., 1984, Mol. Gen. Genet., 197: 345-346). A skilled artisan, therefore, recognizes that such a system allows for insertion of multiple heterologous genes by selecting and counterselecting.

Further Enzymatic Modifications

Further enzymatic deletions may be beneficial or necessary to isolate an Ig free of mannosylphosphorylation or β-mannosylation which may confer aberrant immunogenic activities in humans. As mentioned, U.S. patent application Ser. No. 11/020,808 discloses a method for the elimination of mannosylphosphorylation, and U.S. Pat. application Ser. No. 11/118,008 discloses a method for the elimination of β-mannosylation.

Production of Ig having Predominantly Man₅GlcNAc₂ glycan Structure in Other Protein Expression Systems

It is understood by the skilled artisan that an expression host system (organism) is selected for heterologous protein expression that may or may not need to be engineered to express Igs having a predominant glycan structure. The Examples provided herein are examples of one method for carrying out the expression of Ig with a particular glycan at Asn-297 or another N-glycosylation site, or both. One skilled in the art can easily adapt these details of the invention and examples for any protein expression host system (organism).

Other protein expression host systems including animal, plant, insect, bacterial cells and the like may be used to produce Ig molecules and compositions according to the present invention. Such protein expression host systems may be engineered or selected to express a predominant glycoform or alternatively may naturally produce glycoproteins having predominant glycan structures. Examples of engineered protein expression host systems producing a glycoprotein having a predominant glycoform include gene knockouts/mutations (Shields et al., 2002, JBC, 277: 26733-26740); genetic engineering in (Umaña et al., 1999, Nature Biotech., 17: 176-180) or a combination of both. Alternatively, certain cells naturally express a predominant glycoform—for example, chickens, humans and cows (Raju et al., 2000, Glycobiology, 10: 477-486). Thus, the expression of an Ig glycoprotein or composition having predominantly one specific glycan structure according to the present invention can be obtained by one skilled in the art by selecting at least one of many expression host systems. Further expression host systems found in the art for production of glycoproteins include: CHO cells: Raju WO9922764A1 and Presta WO03/035835A1; hybridroma cells: Trebak et al., 1999, J. Immunol. Methods, 230: 59-70; insect cells: Hsu et al., 1997, JBC, 272:9062-970, and plant cells: Gerngross et al., WO04/074499A2.

Purification of IgG

Methods for the purification and isolation of antibodies are known and are disclosed in the art. See, for example, Kohler & Milstein, (1975) Nature 256:495; Brodeur et al., Monoclonal Antibody Production Techniques and Applications, pp. 51-63, Marcel Dekker, Inc., New York, 1987); Goding, Monoclonal Antibodies: Principles and Practice, pp. 59-104 (Academic Press, 1986); and Jakobovits et al. (1993) Proc. Natl. Acad. Sci. USA 90:2551-255 and Jakobovits et al, (1993) Nature 362:255-258. In a further embodiment, antibodies or antibody fragments can be isolated from antibody phage libraries generated using the techniques described in McCafferty et al. (1990) Nature, 348:552-554 (1990), using the antigen of interest to select for a suitable antibody or antibody fragment.

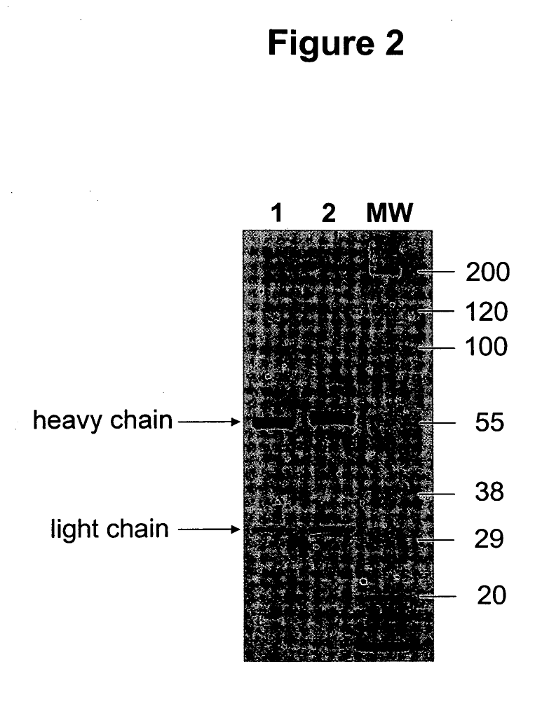

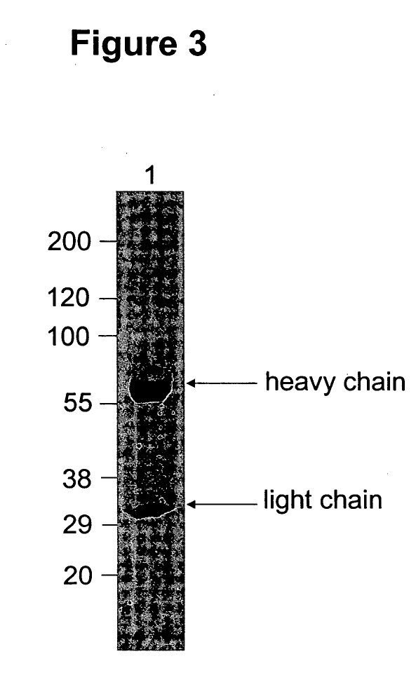

Recombinant Ig molecules produced according to the methods of the present invention can be purified according to methods outlined in Example 3. FIG. 2 shows an SDS-PAGE Coomassie stained gel of JC-IgG purified from YJN531-1. FIG. 3 shows an SDS-PAGE Coomassie stained gel of DX-IgG purified from YGLY14. In another embodiment, the purified Ig antibody has Man₅GlcNAc₂ as the predominant N-glycan. The glycan analysis and distribution on any Ig molecule can be determined by several mass spectroscopy methods known to one skilled in the art, including but not limited to: HPLC, NMR, LCMS and MALDI-TOF MS. In a preferred embodiment, the glycan distribution is determined by MALDI-TOF MS analysis as disclosed in Example 5. FIG. 4A shows a MALDI-TOF spectra of JC-IgG purified from YJN531-1 and treated with α-1,2 mannosidase (Example 3). This MALDI-TOF shows approximately 72 mole % of the total N-glycans are Man₅GlcNAc₂. FIG. 4B shows a MALDI-TOF spectra of DX-IgG purified from YGLY14 and treated with α-1,2 mannosidase. This MALDI-TOF shows approximately 90 mole % of the total N-glycans are Man₅GlcNAc₂.

Pharmaceutical Compositions

Antibodies of the invention can be incorporated into pharmaceutical compositions comprising the antibody as an active therapeutic agent and a variety of other pharmaceutically acceptable components. See Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation can also include other carriers, adjuvants, or nontoxic, nontherapeutic, nonimmunogenic stabilizers and the like.

Pharmaceutical compositions for parenteral administration are sterile, substantially isotonic, pyrogen-free and prepared in accordance with GMP of the FDA or similar body. Antibodies can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249, 1527 (1990) and Hanes, Advanced Drug Delivery Reviews 28, 97-119 (1997).

Diagnostic Products

Antibodies of the invention can also be incorporated into a variety of diagnostic kits and other diagnostic products such as an array. Antibodies are often provided prebound to a solid phase, such as to the wells of a microtiter dish. Kits also often contain reagents for detecting antibody binding, and labeling providing directions for use of the kit. Immunometric or sandwich assays are a preferred format for diagnostic kits (see U.S. Pat. Nos. 4,376,110, 4,486,530, 5,914,241, and 5,965,375). Antibody arrays are described by e.g., U.S. Pat. No. 5,922,615, U.S. Pat. No. 5,458,852, U.S. Pat. No. 6,019,944, and U.S. Pat. No. 6,143,576.

Therapeutic Applications

The present invention provides glycoprotein compositions which comprise predominantly a particular glycoform on the glycoprotein. It is a feature of the present invention that when administered to mammals including humans, pharmaceutical compositions comprising the novel glycoprotein compositions, in preferred embodiments, advantageously exhibit superior in vivo properties when compared to other glycoprotein compositions having similar primary structure. Thus, the novel compositions of the invention may be used wherever the glycoprotein pharmaceutical agent is presently used and may advantageously provide improved properties as well as increased uniformity between and throughout production lots. The preparations of the invention can be incorporated into solutions, unit dosage forms such as tablets and capsules for oral delivery, as well as into suspensions, ointments and the like, depending on the particular drug or medicament and its target area.

In a particular aspect, the present invention provides novel compositions for glycoprotein pharmaceutical agents, drugs or medicaments wherein the glycoprotein comprises an immunoglobulin molecule and the composition comprises predominantly particular glycoforms of the glycoprotein agent. According to a particular aspect of the invention, compositions are provided comprising an immunoglobulin glycoprotein having predominantly an N-linked oligosaccharide of the Man₅GlcNAc₂ glycan structure as described herein. In preferred aspects, the glycoprotein is an antibody and especially may be a monoclonal antibody. The invention further provides methods and tools for producing the compositions of the invention.

The invention further encompasses pharmaceutical compositions comprising the glycoform preparations of the invention. The compositions are preferably sterile. Where the composition is an aqueous solution, preferably the glycoprotein is soluble. Where the composition is a lyophilized powder, preferably the powder can be reconstituted in an appropriate solvent.

In other aspects, the invention involves a method for the treatment of a disease state comprising administering to a mammal in need thereof a therapeutically effective dose of a pharmaceutical composition of the invention. It is a further object of the invention to provide the glycoform preparations in an article of manufacture or kit that can be employed for purposes of treating a disease or disorder.

The Ig molecules of the present invention having predominantly Man₅GlcNAc₂ N-glycans have many therapeutic applications for indications such as cancers, inflammatory diseases, infections, immune diseases, autoimmune diseases including idiopathic thrombocytopenic purpura, arthritis, systemic lupus erythrematosus, and autoimmune hemolytic anemia.

The following are examples which illustrate the compositions and methods of this invention with reference to production of an Ig glycoprotein composition. These examples should not be construed as limiting—the examples are included for the purposes of illustration only. The skilled artisan recognizes that numerous modifications and extensions of this disclosure including optimization are possible. Such modifications and extensions are considered part of the invention.

### EXAMPLE 1

Cloning of DX-IgG1 for Expression in P. pastoris

The light (L) and heavy (H) chains of DX-IgG 1 (an anti-CD20 IgG 1) consists of mouse variable regions and human constant regions. The light chain is disclosed as SEQ ID NO: 1 and heavy chain as SEQ ID NO: 2. The heavy and light chain sequences were synthesized using overlapping oligonucleotides purchased from Integrated DNA Technologies (IDT). For the light chain variable region, 15 overlapping oligonucleotides (SEQ ID NOs: 5-19) were purchased and annealed using Extaq (Takada) in a PCR reaction to produce the light chain variable region fragment having a 5′ MlyI site. This light chain variable fragment was then joined with the light chain constant region (SEQ ID NO: 3) (Gene Art, Toronto, Canada) by overlapping PCR using the 5′ MlyI primer CD20L/up (SEQ ID NO: 20), the 3′ variable/5′ constant primer LfusionRTVAAPS/lp (SEQ ID NO: 21), the 3′ constant region primer Lfusion RTVAAPS/lp (SEQ ID NO: 22) and 3′CD20L/lp (SEQ ID NO: 23). The final MlyI-light chain fragment (which included 5′AG base pairs) was then inserted into pCR2.1 topo vector (Invitrogen) resulting in pDX343. For the heavy chain, 17 overlapping oligonucleotides (SEQ ID NOs: 24-40) corresponding to the mouse heavy chain variable region were purchased from IDT and annealed using Extaq. This heavy chain variable fragment was then joined with the heavy chain constant region (SEQ ID NO: 4) (Gene Art) by overlapping PCR using the 5′ MlyI primer CD20H/up (SEQ ID NO: 41), the 5′ variable/constant primer HchainASTKGPS/up (SEQ ID NO: 42), the 3′ variable/constant primer HchainASTKGPS/lp (SEQ ID NO: 43) and the 3′ constant region primer HFckpnl/lp (SEQ ID NO: 44). The final MlyI-heavy chain fragment (which included 5′AG base pairs) was inserted into pCR2.1 topo vector (Invitrogen) resulting in pDX360. The full length light chain and full length heavy chain were isolated from the respective topo vectors as Mly1 and Not1 fragments. These light chain and heavy chain fragments were then ligated to a Kar2(Bip) signal sequence (SEQ ID NO: 45) using 4 overlapping oligonucleotides—P.BiPss/UP1-EcoRI, P.BiPss/LP1, P.BiPss/UP2 and P.BiP/LP2 (SEQ ID NOS: 4649, respectively), and then ligated into the EcoRI-NotI sites of pPICZA resulting in pDX344 carrying the Kar2-light chain and pDX468 carrying the Kar2-heavy chain. A BglII-BamHI fragment from pDX344 was then subcloned into pBK85 containing the AOX2 promoter gene for chromosomal integration, resulting in pDX458. A BglII-BamHI fragment from pDX468 carrying the heavy chain was then subcloned into pDX458, resulting in pDX478 containing both heavy and light chains of CD20 under the AOX1 promoter. This plasmid was then linearized with SpeI prior to transformation for integration into the AOX2 locus with transformants selected using Zeocin resistance (see Example 2).

Cloning of JC-IgG for Expression in P. pastoris

The light (L) and heavy (H) chains of the JC-IgG1 consists of mouse variable regions and human constant regions. The mouse variable light chain is disclosed as SEQ ID NO: 50 (GenBank #AF013576) and mouse variable heavy chain as SEQ ID NO: 51 (GenBank #AF013577). The heavy and light chain sequences were synthesized using overlapping oligonucleotides purchased from Integrated DNA Technologies (IDT). For the light chain, 12 overlapping oligonucleotides (SEQ ID NOs: 52-63) were purchased and annealed using Extaq (Takada) in a PCR reaction to produce the 660 base pair light chain having a 5′ EcoRI site and a 3′ Kpn1 site. This light chain was then subcloned into a pPICZa vector (Invitrogen) as an EcoRI-KpnI fragment. For the heavy chain, 12 overlapping oligonucleotides (SEQ ID NOs: 64-75) corresponding to the Fab fragment were purchased and annealed using Extaq to produce the 660 base pair Fab fragment. The Fc fragment was synthesized using 12 overlapping oligonucleotides (SEQ ID NOs: 76-87) which were annealed in a similar overlapping PCR reaction. Both Fab and Fc fragments of the heavy chain were then annealed using a 5′ EcoRI primer (SEQ ID NO: 64) corresponding to the 5′ end of the heavy Fab fragment and a 3′ Kpn1 primer (SEQ ID NO: 88) corresponding to the 3′ end of the Fc fragment using pFU Turbo polymerase (Stratagene) producing the 1,330 base pair heavy chain. Using 5′ EcoRI and 3′ Kpn1 sites encoded in the primers, the heavy chain was cloned into a pPICZa vector. The AOX2 promoter sequence, which functions as an integration locus, was subcloned into a final pPICZa vector. Next, a BglII-BstB1 fragment containing the AOX1 promoter and a BstB1-BamHI fragment containing an HSA sequence from a human liver cDNA library (SEQ ID NO:89), thrombin site (SEQ ID NO:90) and JC light chain were both subcloned into the BamHI site of this AOX2/pPICZa vector. Then another a BlgII-BstBI fragment containing the AOX1 promoter and a BstIB1-BamHI fragment containing an HSA sequence, thrombin site and JC heavy chain were subcloned into the BamHI site of this same pPICZa vector. This final vector contains the AOX2 integration locus, HSA-tagged JC light chain and HSA-tagged JC heavy chain, resulting in pJC140. This expression cassette was integrated into the AOX2 locus of a P. pastoris strain with transformants selected for zeocin resistance. (See Example 2.)

Rituximab®/Rituxan® is an Anti-CD20 Mouse/Human Chimeric IgG1 Purchased from Biogen-IDEC/Genentech, San Francisco, Calif.

PCR amplification. An Eppendorf Mastercycler was used for all PCR reactions. PCR reactions contained template DNA, 125 μM dNTPs, 0.2 μM each of forward and reverse primer, Ex Taq polymerase buffer (Takara Bio Inc.), and Ex Taq polymerase or pFU Turbo polymerase buffer (Stratagene) and pFU Turbo polymerase. The DNA fragments were amplified with 30 cycles of 15 sec at 97° C., 15 sec at 55° C. and 90 sec at 72° C. with an initial denaturation step of 2 min at 97° C. and a final extension step of 7 min at 72° C.

PCR samples were separated by agarose gel electrophoresis and the DNA bands were extracted and purified using a Gel Extraction Kit from Qiagen. All DNA purifications were eluted in 10 mM Tris, pH 8.0 except for the final PCR (overlap of all three fragments) which was eluted in deionized H₂O.

### EXAMPLE 2

Transformation of IzG vectors into P. pastoris strain YJN531-1 and YGLY14. The vector DNA is prepared by adding sodium acetate to a final concentration of 0.3 M. One hundred percent ice cold ethanol is then added to a final concentration of 70% to the DNA sample. The DNA is pelleted by centrifugation (12000 g×10 min) and washed twice with 70% ice cold ethanol. The DNA is dried and resuspended in 50 μl of 10 mM Tris, pH 8.0. A YJN531-1 or YGLY14 yeast culture (Choi et al., 2003; Hamilton et al., 2003) to be transformed is prepared by expanding a smaller culture in BMGY (buffered minimal glycerol: 100 mM potassium phosphate, pH 6.0; 1.34% yeast nitrogen base; 4×10⁻⁵% biotin; 1% glycerol) to an O.D. of ˜2-6. The yeast cells are then made electrocompetent by washing 3 times in 1M sorbitol and resuspending in ˜1-2 mls 1M sorbitol. DNA (1-2 μg) is mixed with 100 μl of competent yeast and incubated on ice for 10 min. Yeast cells are then electroporated with a BTX Electrocell Manipulator 600 using the following parameters; 1.5 kV, 129 ohms, and 25 μF. One milliliter of YPDS (1% yeast extract, 2% peptone, 2% dextrose, 1M sorbitol) was added to the electroporated cells. Transformed yeast was subsequently plated on selective agar plates containing zeocin (see Example 3).

Culture conditions for IgG1 in P. pastoris A single colony of YGLY14 transformed with pDX478 or YJN531-1 transformed with pJC140 was inoculated into 10 ml of BMGY media (consisting of 1% yeast extract, 2% peptone, 100 mM potassium phosphate buffer (pH 6.0), 1.34% yeast nitrogen base, 4×10⁻⁵% biotin, and 1% glycerol) in a 50 ml Falcon Centrifuge tube. The culture was incubated while shaking at 24° C./170-190 rpm for 48 hours until the culture was saturated. 100 ml of BMGY was then added to a 500 ml baffled flask. The seed culture was then transferred into a baffled flask containing the 100 ml of BMGY media. This culture was incubated with shaking at 24° C./170-190 rpm for 24 hours. The contents of the flask was decanted into two 50 ml Falcon Centrifuge tubes and centrifuged at 300 rpm for 10 minutes. The cell pellet was washed once with 20 ml of BMGY without glycerol, followed by gentle resuspension with 20 ml of BMMY (BMGY with 1% MeOH instead of 1% glycerol). The suspended cells were transferred into a 250 ml baffled flask. The culture was incubated with shaking at 24° C./170-190 rpm for 24 hours. The contents of the flask was then decanted into two 50 ml Falcon Centrifuge tubes and centrifuged at 3000 rpm for 10 minutes. The culture supernatant was analyzed by ELISA to determine approximate antibody titer prior to protein isolation (see Example 3). Quantification of antibody in culture supernatants was performed by enzyme linked immunosorbent assays (ELISAs): High binding microtiter plates (Costar) were coated with 24 μg of goat anti-human Fab (Biocarta, Inc, San Diego, Calif.) in 10 ml PBS, pH 7.4 and incubate over night at 4° C. Buffer was removed and blocking buffer (3% BSA in PBS), was added and then incubated for 1 hour at room temperature. Blocking buffer was removed and the plates were washed 3 times with PBS. After the last wash, increasing volume amounts of antibody culture supernatant (0.4, 0.8, 1.5, 3.2, 6.25, 12.5, 25 and 50 μl) was added and incubated for 1 hour at room temperature. Plates were then washed with PBS+0.05% Tween20. After the last wash, anti-human Fc-HRP was added in a 1:2000 PBS solution, and then incubated for 1 hour at room temperature. Plates were then washed 4 times with PBS-Tween20. Plates were analyzed using TMB substrate kit following manufacturer's instructions (Pierce Biotechnology).

### EXAMPLE 3

Purification of IgG1

Monoclonal antibodies were captured from the culture supernatant using a Streamline Protein A column. Antibodies were eluted in Tris-Glycine pH 3.5 and neutralized using 1M Tris pH 8.0. Further purification was carried out using hydrophobic interaction chromatography (HIC). The specific type of HIC column depends on the antibody. For the JC-IgG and the DX-IgG a phenyl sepharose column (can also use octyl sepharose) was used with 20 mM Tris (7.0), 1M (NH₄)₂SO₄ buffer and eluted with a linear gradient buffer of 1M to 0M (NH₄)₂SO₄. The antibody fractions from the phenyl sepharose column were pooled and exchanged into 50 mM NaOAc/Tris pH 5.2 buffer for final purification through a cation exchange (SP Sepharose Fast Flow) (GE Healthcare) column. Antibodies were eluted with a linear gradient using 50 mM Tris, 1M NaCl (pH 7.0)

Treatment of Ig-Man₈GlcNAc₂ with α-1,2 Mannosidase

For α1,2 mannosidase treatment, 5 mg of purified IgG-Man₈GlcNAc₂ (JC-IgG or DX-IgG) is buffer exchanged into 50 mM NH₄Ac pH 5.0. In a siliconized tube, and 0.03 U α-1,2 mannosidase (EMD Biosciences, La Jolla, Calif.) was added to the purified IgG in 50 mM NH₄Ac pH 5.0 and incubated for 16-24 hours at 37° C. A sample of this is evaporated to dryness, resuspended in water and analyzed by MALDI-TOF. The antibody was then purified from the α-1,2 mannosidase following a phenyl sepharose purification as described above.

### EXAMPLE 4

Detection of Purified Ig

Purified JC-IgG or DX-IgG were mixed with an appropriate volume of sample loading buffer and subjected to sodium dodecyl sulfate-polyacrylamide gel electrophoresis (SDS-PAGE) with precast gels according to the manufacturer's instructions (NuPAGE bis-Tris electrophoresis system; Invitrogen Corporation, Carlsbad, Calif.). The gel proteins were stained with Coomassie brilliant blue stain (Bio-Rad, Hercules, Calif.). See FIGS. 2 and 3.

Antibody Concentrations

The concentration of protein chromatography fractions were determined using a Bradford assay (Bradford, M. 1976, Anal. Biochem. (1976) 72, 248-254) using albumin as a standard (Pierce, Rockford, Ill.)

### EXAMPLE 5

IgG1 Carbohydrate Analysis

Matrix Assisted Laser Desorption Ionization Time of Flight Mass Spectrometry (MALDI-TOF MS). MALDI-TOF analysis of aspargine-linked oligosaccharides: N-linked glycans were released from JC-IgG and DX-IgG using a modified procedure of Papac et al., Glycobiology 8, 445-454 (1998). A sample of the antibodies was reduced and carboxymethylated and the membranes were blocked, the wells were washed three times with water. The IgG proteins were deglycosylated by the addition of 30 ul of 10 mM Nh4HCO3 (pH 8.3) containing 1 mU of N-glycanase (EMD Biosciences, La Jolla, Calif.). After 16 hours at 37° C., the solution containing the glycans was removed by centrifugation and evaporated to dryness. The dried glycans from each well were dissolved in 15 μl of water, and 0.5 μl was spotted on stainless-steel sample plates and mixed with 0.5 μl of S-DHB matrix (9 mg/ml of dihydroxybenzoic acid/1 mg/ml of 5-methoxy-salicylic acid in 1:1 water/acetonitrile/0.1% trifluoroacetic acid) and allowed to dry. Ions were generated by irradiation with a pulsed nitrogen laser (337 nm) with a 4-ns pulse time. The instrument was operated in the delayed extraction mode with a 125-ns delay and an accelerating voltage of 20 kV. The grid voltage was 93.00%, guide wire voltage was 0.1%, the internal pressure was <5×10⁻⁷ torr (1 torr=133 Pa), and the low mass gate was 875 Da. Spectra were generated from the sum of 100-200 laser pulses and acquired with a 500-MHz digitizer. (Man)₅(GlcNAc)₂ oligosaccharide was used as an external molecular weight standard. All spectra were generated with the instrument in the positive-ion mode.

### EXAMPLE 6

Antigen Binding ELISA Assay

High binding microtiter plates (Costar) were coated with 10 ug of antigen in PBS, pH 7.4 and incubate over night at 4° C. Buffer was removed and blocking buffer (3% BSA in PBS), was added and then incubated for 1 hour at room temperature. Blocking buffer was removed and the plates were washed 3 times with PBS. After the last wash, increasing amounts of purified antibody were added from 0.2 ng to 100 ng and incubated for 1 hour at room temperature. Plates were then washed with PBS+0.05% Tween20. After the last wash, anti-human Fc-HRP was added in a 1:2000 PBS solution, and then incubated for 1 hour at room temperature. Plates were then washed 4 times with PBS-Tween20. Plates were analyzed using TMB substrate kit following manufacturer's instructions (Pierce Biotechnology).

Fc Receptor Binding Assays

Fc receptor binding assays for FcγRIIb, FcγRIIIa and FcγRIIIb were carried out according to the protocols previously described (Shields et al., 2001, J. Biol. Chem, 276: 6591-6604). For FcγRIII binding: FcγRIIIb (FIG. 5) and FγRIIb (FIG. 7) fusion proteins at 1 μg/ml or FcγRIIIa-LF (FIG. 6) fusion proteins at 0.8 μg/m in PBS, pH 7.4, were coated onto ELISA plates (Nalge-Nunc, Naperville, Ill.) for 48 h at 4° C. Plates were blocked with 3% bovine serum albumin (BSA) in PBS at 25° C. for 1 h. JC-IgG or DX-IgG dimeric complexes were prepared in 1% BSA in PBS by mixing 2:1 molar amounts of JC-IgG or DX-IgG and HRP-conjugated F(Ab′)2anti-F(Ab′)₂ at 25° C. for 1 h. Dimeric complexes were then diluted serially at 1:2 in 1% BSA/PBS and coated onto the plate for 1 hour at 25° C. The substrate used is 3,3′,5,5′-tetramethylbenzidine (TMB) (Vector Laboratories). Absorbance at 450 nm was read following instructions of the manufacturer (Vector Laboratories).

ELISPOT Assay for Antibody Feedback in B Cells.

This assay is conducted as described in Westman, et al., 1997, Scand. J. Immunol. 46: 10-15. BSA (bovine serum albumin) is first conjugated to an IgG antibody resulting in a BSA-IgG complex. The number of B cells secreting BSA-specific IgG is determined using an ELISPOT assay. Spleens are removed from injected mice and cell suspensions are prepared in DMEM (Gibco, New York) with 0.5% normal mouse serum. One hundered microliter cell suspensions are applied to BSA-coated microtiter plates (see ELISA protocol above) and incubated at 37° C., 5% CO₂ for 3.5 h. Plates are washed and incubated at 4° C. o.n. with 50 μl of alkaline phosphatase-conjugated sheep anit-mouse IgG dilute 1/100 in PBS-Tween. Spots are developed for 1 hour at room temperature in 50 μl of 5 bromo4-chloro-3-indoyl phosphate (Sigma-Aldrich) and counted under a stereomicroscope.

### EXAMPLE 7

For ADCC assayed using a blood matrix study (e.g. B-cell depletion) as described in Vugmeyster and Howell, 2004, Int. Immunopharm. 4: 1117-1124. Whole blood depleted of plasma and red blood cells (RBCs) is reconstituted in stain buffer (Hank's balanced salt solution (HBSS) with 1% BSA and 0.1% sodium azide) leading to leukocyte suspension in stain buffer. Whole blood sample is then spun for 5 minutes at 1000 g, the supernatant (plasma) is discarded and the pellet is treated with ammonium chloride lysing (ACL) reagent, washed, and resuspended in an equivalent volume of stain buffer. For B-cell depletion assay: 10 μl of 100 μg/ml solution of antibody or stain buffer is added to 90 μl of SB matrix and incubated for 1 hour at 37° C. Samples are stained immediately with anti-CD19-FITC and anti-CD45-PE for 30 minutes at 25° C. Samples are then fixed in 1% formaldehyde and run in triplicate. Quantification of B-cell depletion is obtained by flow cytometry. Flow cytometric analysis of B-cell depletion: A FACS Calibur (BD Biosciences) instrument equipped with an automated FACS Loader and Cell Quest Software is used for acquisition and analysis of all samples. Cytometer QC and setup include running CaliBrite beads and SpheroTech rainbow beads (BD Biosciences) to confirm instrument functionality and detector linearity. Isotype and compensation controls are run with each assay to confirm instrument settings. Percent of B cells of total lymphocytes is obtained by the following gating strategy. The lymphocyte population is marked on the forward scatter/side scatter scattegram to define Region 1 (R1). Using events in R1, fluorescence intensity dot plots are displayed for CD19 and CD45 markers. Fluorescently labeled isotype controls are used to determine respective cutoff points for CD19 and CD45 positivity. % B is determined using CellQuest as a fraction of cells in R1 region that have CD19-positive, CD45-positive phenotype. Triplicate samples are run for each treatment group. The percent B cell depletion is calculated using the formula average [100*(1−% B treated with control antibody/average [% B treated with SB]). Fluorescent dye release ADCC assay: PBMC isolation: Peripheral venous blood from healthy individuals or blood donors (10-20) is collected into heparinised vacutainer tubes (Becton Dickinson Vacutainer Systems, Rutherford, N.J., USA). Approximately 5 ml of blood is required for implanting 2 mice. Peripheral blood mononuclear cells (PBMCs) are separated by centrifugation using OptiPrep following manufacturer's instructions. PBMCs are washed once with complete culture media (CM) consisting of RPMI 1640, 2 mM L-glutamine, 100 IU/ml penicillin, 100 g/ml streptomycin (Gibco/BRL) and supplemented with 20% fetal calf serum, and then resuspended at a concentration of 1×10⁶/ml CM and transfered to a 250 ml culture flask (Falcon, N.J., USA) for monocyte depletion. After 1 hour of incubation at 37° C. and 5% CO₂, non-adherent cells are recovered, washed once with culture media and the peripheral blood lymphocytes (PBLs) are adjusted to a concentration of 2.5×10⁷/ml CM. Fluorescent dye-release ADCC. The premise behind the ADCC assay is that antibody binding to CD20 or CD40 antigen presenting target cells (Raji cell line or BCL1-3B3 cells, respectively) stimulates target cell binding to Fcγ receptors on the effector cells. This in turn promotes lysis of the target cells presenting the CD20 or CD40 antigen, releasing an internal fluorescent dye that can be quantified. Alamar-blue fluorescence is used in place of ⁵¹Cr labeling of the target cells. 50 ul of CD20-presenting Raji cell suspension (1×10⁴ cells) is combined with 50 ul amount of anti-DX-IgG or anti-JC-IgG mAb (various concentrations) and 50 ul amount of PBMC effector cells isolated as described above (effector to target cell ratio can be 100:1, 50:1. 25:1 and 12.5:1) in 96 well tissue culture plates and incubated for 4 h hours at 37 temperature and 5% CO₂ to facilitate lysis of the Raji or BCL1-3B3 cells. 50 μl of Alamar blue is added and the incubation is continued for another 5 hours to allow for uptake and metabolism of the dye into its fluorescent state. The plates cool to room temperature on a shaker and the fluorescence is read in a fluorometer with excitation at 530 nm and emission at 590 nm. Relative fluorescence units (RFU) are plotted against mAb concentrations and sample concentrations are computed from the standard curve using a control antibody—e.g. Rituximab®. In vivo ADCC using Severe Combined Immunodeficient (SCID) mice (Niwa et al., 2004, Cancer Research, 64: 2127-2133). In vivo ADCC activity can be assayed using a mouse model engrafted with human peripheral blood mononuclear cells (PMBCs) from healthy donors which include heterozygous (FcγRIIIa-LF/FcγRIIIa-LV) and homozygous (FcγRIIIa-LV/FcγRIIIa-LV and FcγRIIIa-LF/FcγRIIIa-LF) genotypes. Using this model system, Igs having a predominant N-glycan are assayed for enhanced ADCC activity compared with Rituximab® or any other control antibody. A detailed and sufficient protocol for this in vivo ADCC assay is found in Niwa et al., 2004, supra.

SEQ ID NO: 01
(mouse/human chimeric IgG1 light chain)
caaatcgtcttgtctcaatccccagctattttgtctgcttcccctggagagaaggtcaccatgacttgtagagcctcttcctctg
tctcttacattcactggttccagcaaaagccaggttcctctccaaagccatggatcacgctacttccaacttggcttccggtgtt
ccagttagattctctggttctggttccggtacctcctactctcttaccatctccagagttgaagccgaggacgctgctacttact
actgtcagcaatggacttctaacccaccaactttcggtggtggtaccaaattggagattaagagaactgttgctgctccatccgt
tttcattttcccaccatccgacgaacaattgaagtctggtacagcttccgttgtttgtttgttgaacaacttctacccaagagag
gctaaggttcagtggaaggttgacaacgctttgcaatccggtaactcccaagaatccgttactgagcaggattctaaggattcca
cttactccttgtcctccactttgactttgtccaaggctgattacgagaagcacaaggtttacgcttgtgaggttacacatcaggg
tttgtcctccccagttactaagtccttcaacagaggagagtgttaa
SEQ ID NO: 02
(mouse/human chimeric IgG1 heavy chain)
caagtccagttgcaacagcctggtgccgagttggtcaagccaggtgcttctgttaagatgtcctgtaaggcttctggttacactt
tcacctcctacaacatgcactgggtcaagcaaactccaggtagaggtttggagtggttggtgccatctacccaggtaacggtgac
acttcttacaaccaaaaattcaagggaaaggctactcttaccgctgataagtcctcttccaccgcctacatgcaattgtcttcct
tgacttctgaagattctgctgtttactactgtgctagatccacctactacggtggagactggtacttcaacgtttggggtgctgg
taccactgtcaccgtttccgctgcttctactaagggaccatccgtttttccattggctccatcctctaagtctacttccggtggt
actgctgctttgggatgtttggttaaggactacttcccagagcctgttactgtttcttggaactccggtgctttgacttctggtg
ttcacactttcccagctgttttgcaatcttccggtttgtactccttgtcctccgttgttactgttccatcctcttccttgggtac
tcagacttacatctgtaacgttaaccacaagccatccaacactaaggttgacaagaaggctgagccaaagtcctgtgacaagaca
catacttgtccaccatgtccagctccagaattgttgggtggtccatccgttttcttgttcccaccaaagccaaaggacactttga
tgatctccagaactccagaggttacatgtgttgttgttgacgtttctcacgaggacccagaggttaagttcaactggtacgttga
cggtgttgaagttcacaacgctaagactaagccaagagaggagcagtacaactccacttacagagttgtttccgttttgactgtt
ttgcaccaggattggttgaacggaaaggagtacaagtgtaaggtttccaacaaggctttgccagctccaatcgaaaagactatct
ccaaggctaagggtcaaccaagagagccacaggtttacactttgccaccatccagagatgagttgactaagaaccaggtttcctt
gacttgtttggttaaaggattctacccatccgacattgctgttgagtgggaatctaacggtcaaccagagaacaactacaagact
actccaccagttttggattctgacggttccttcttcttgtactccaagttgactgttgacaagtccagatggaacagggtaacgt
tttctcctgttccgttatgcatgaggctttgcacaaccactacactcaaaagtccttgtctttgtccccaggtaagtaa
SEQ ID NO: 03
(light constant region of human IgG1)
agaactgttgctgctccatccgttttcattttcccaccatccgacgaacaattgaagtctggtacagcttccgttgtttgtttgt
tgaacaacttctacccaagagaggctaaggttcagtggaaggttgacaacgctttgcaatccggtaactcccaagaatccgttac
tgagcaggattctaaggattccacttactccttgtcctccactttgactttgtccaaggctgattacgagaagcacaaggtttac
gcttgtgaggttacacatcagggtttgtcctccccagttactaagtccttcaacagaggagagtgttaa
SEQ ID NO: 04
(heavy constant region of human IgG1)
tctactaagggaccatccgtttttccattggctccatcctctaagtctacttccggtggtactgctgctttgggatgtttggtta
aggactacttcccagagcctgttactgtttcttggaactccggtgctttgacttctggtgttcacactttcccagctgttttgca
atcttccggtttgtactccttgtcctccgttgttactgttccatcctcttccttgggtactcagacttacatctgtaacgttaac
cacaagccatccaacactaaggttgacaagaaggctgagccaaagtcctgtgacaagacacatacttgtccaccatgtccagctc
cagaattgttgggtggtccatccgttttcttgttcccaccaaagccaaaggacactttgatgatctccagaactccagaggttac
atgtgttgttgttgacgtttctcacgaggacccagaggttaagttcaactggtacgttgacggtgttgaagttcacaacgctaag
actaagccaagagaggagcagtacaactccacttacagagttgtttccgttttgactgttttgcaccaggattggttgaacggaa
aggagtacaagtgtaaggtttccaacaaggctttgccagctccaatcgaaaagactatctccaaggctaagggtcaaccaagaga
gccacaggtttacactttgccaccatccagagatgagttgactaagaaccaggtttccttgacttgtttggttaaaggattctac
ccatccgacattgctgttgagtgggaatctaacggtcaaccagagaacaactacaagactactccaccagttttggattctgacg
gttccttcttcttgtactccaagttgactgttgacaagtccagatggaacagggtaacgttttctcctgttccgttatgcatgag
gctttgcacaaccactacactcaaaagtccttgtctttgtccccaggtaagtaa
SEQ ID NO: 05
(CD20LF1)
aggagtcgtattcaaatcgtcttgtctcaatccccagctattttg
SEQ ID NO: 06
(CD20LF2)
tctgcttcccctggagagaaggtcaccatgacttgtagagcctct
SEQ ID NO: 07
(CD20LF3)
tcctctgtctcttacattcactggttccagcaaaagccaggttcc
SEQ ID NO: 08
(CD20LF4)
tctccaaagccatggatctacgctacttccaacttggcttccggt
SEQ ID NO: 09
(CD20LF5)
gttccagttagattctctggttctggttccggtacctcctactct
SEQ ID NO: 10
(CD20LF6)
cttaccatctccagagttgaagccgaggacgctgctacttactac
SEQ ID NO: 11
(CD20LF7)
tgtcagcaatggacttctaacccaccaactttcggtggtggtacc
SEQ ID NO: 12
(CD20LF8)
aaattggagattaagagaactgttgctgctccatcc
SEQ ID NO: 13
(CD20LR1)
caacagttctcttaatctccaatttggtaccaccaccgaaagttg
SEQ ID NO: 14
(CD20LR2)
gtgggttagaagtccattgctgacagtagtaagtagcagcgtcct
SEQ ID NO: 15
(CD20LR3)
cggcttcaactctggagatggtaagagagtaggaggtaccggaac
SEQ ID NO: 16
(CD20LR4)
agaaccagagaatctaactggaacaccggaagccaagttggaag
SEQ ID NO: 17
(CD20LR5)
tagcgtagatccatggctttggagaggaacctggcttttgctgga
SEQ ID NO: 18
(CD20LR6)
ccagtgaatgtaagagacagaggaagaggctctacaagtcatgg
SEQ ID NO: 19
(CD20LR7)
tgaccttctctccaggggaagcagacaaaatagctggggattgag
SEQ ID NO: 20
(CD20L/up)
aggagtcgtattcaaatcgtc
SEQ ID NO: 21
(LfusionRTVAAPS/up)
agaactgttgctgctccatcc
SEQ ID NO: 22
(LfusionRTVAAPS/lp)
ggatggagcagcaacagttc
SEQ ID NO: 23
(CD20L/lp)
ctggtaccttaacactctcctctgttgaag
SEQ ID NO: 24
(CD20HF1)
aggagtcgtattcaagtccagttgcaacagcctggtgccgagttg
SEQ ID NO: 25
(CD20HF2)
gtcaagccaggtgcttctgttaagatgtcctgtaaggcttctggt
SEQ ID NO: 26
(CD20HF3)
tacactttcacctcctacaacatgcactgggtcaagcaaactcca
SEQ ID NO: 27
(CD20HF4)
ggtagaggtttggagtggattggtgccatctacccaggtaacggt
SEQ ID NO: 28
(CD20HF5)
gacacttcttacaaccaaaaattcaagggaaaggctactcttacc
SEQ ID NO: 29
(CD20HF6)
gctgataagtcctcttccaccgcctacatgcaattgtcttccttg
SEQ ID NO: 30
(CD20HF7)
acttctgaagactctgctgtttactactgtgctagatccacctac
SEQ ID NO: 31
(CD20HF8)
acggtggagactggtacttcaacgtttggggtgctggtaccact
SEQ ID NO: 32
(CD20HF9)
gtcaccgtttccgctgcttctactaagggaccatcc
SEQ ID NO: 33
(CD20HR1)
tagtagaagcagcggaaacggtgacagtggtaccagcaccccaaa
SEQ ID NO: 34
(CD20HR2)
cgttgaagtaccagtctccaccgtagtaggtggatctagcacag
SEQ ID NO: 35
(CD20HR3)
agtaaacagcagagtcttcagaagtcaaggaagacaattgcatgt
SEQ ID NO: 36
(CD20HR4)
aggcggtggaagaggacttatcagcggtaagagtagcctttccct
SEQ ID NO: 37
(CD20HR5)
tgaatttttggttgtaagaagtgtcaccgttacctgggtagatgg
SEQ ID NO: 38
(CD20HR6)
caccaatccactccaaacctctacctggagtttgcttgacccagt
SEQ ID NO: 39
(CD20HR7)
gcatgttgtaggaggtgaaagtgtaaccagaagccttacaggaca
SEQ ID NO: 40
(CD20HR8)
tcttaacagaagcacctggcttgaccaactcggcaccaggctgtt
SEQ ID NO: 41
(CD20H/up)
Aggagtcgtattcaagtccag
SEQ ID NO: 42
(HchainASTKGPs/up)
gcttctactaagggaccatcc
SEQ ID NO: 43
(HchainASTKGPs/lp)
ggatggtcccttagtagaagc
SEQ ID NO: 44
(HFckpn1/lp)
ctggtattacttacctggggacaaagac
SEQ ID NO: 45
(Kar2 signal sequence with EcoRI)
gaattcgaaacgatgctgtcgttaaaaccatcttggctgactttggcggcattaatgtatgccatgctattggtcgtagtgccat
ttgctaaacctgttagagct
SEQ ID NO: 46
(P.BiPss/UP1-EcoRI)
aattcgaaacgatgctgtctttgaagccatcttggcttactttggctgctttgatgtacgctatgctttt
SEQ ID NO: 47
(P.BiPss/LP1)
ccaaagtaagccaagatggcttcaaagacagcatcgtttcg
SEQ ID NO: 48
(P.BiPss/UP2)
ggttgttgttccatttgctaagccagttagagct
SEQ ID NO: 49
(P.BiPss/LP2)
agctctaactggcttagcaaatggaacaacaaccaaaagcatagcgtacatcaaagcag
SEQ ID NO: 50
(GenBank #AF013576)
gatgctgttatgactcaaaacccattgtctttgcctgtttctcttggtgatgaagcttctatttcttgtagatcctctcaatctt
tggaaaactctaacggtaacactttcttgaactggttctttcagaagccaggtcaatctccacaattgttgatttacagagtttc
taacagattttctggtgttccagatagattttctggttctggttctggtactgaacactttgaagatttctagagttgaagctga
agatttgggtgtttacttctgtttgcaagttactcatgttccatacacttttggtggtggtactactttggaaattaagagaact
gttgctgctccatctgtcttcatctttccaccatctgatgaacaattgaagtctggtactgcttctgttgtttgtcttcttaaca
acttctacccaagagaagctaaggttcagtggaaggttgataacgctttgcaatctggtaactctcaagaatctgttactgaaca
agattctaaggattctacttactctttgtcttctactttgactttgtctaaggctgattacgaaaagcataaggtttacgcttgt
gaagttactcatcaaggtttgtcttctccagttactaagtcctttaacagaggtgaatgttag
SEQ ID NO: 51
(GenBank #AF013577)
gatattcaattgcaacaatctggtccaggtttggttaagccatctcaatctttgtctttgacttgttctgttactggttactcta
ttactactaactacaactggaactggattagacaatttccaggtaacaagttggaatggatgggttacattagatacgatggtac
ttctgaatacaccccatctttgaagaacagagtttctattactagagatacttctatgaaccaattcttcttgagattgacttct
gttactccagaagatactgctacttactactgtgctagattggattactggggtcaaggtacttctgttactgtttcttctgctt
ctactaagggtccatctgtttttccacttgctccatcttctaagtctacttctggtggtactgctgctttgggttgtttggttaa
ggattactttccagaaccagttactgtttcttggaactctggtgctttgacttctggtgttcatacttttccagctgttttgcaa
tcttctggtttgtactctttgtcttctgttgttactgttccatcttcttctttgggtactcaaacttacatttgtaacgttaacc
ataagccatctaacactaaggttgataagagagttgaaccaaaatcttgtgataaaactcatacatgtccaccatgtccagctcc
tgaacttctgggtggaccatcagttttcttgttcccaccaaaaccaaaggatacccttatgatttctagaactcctgaagtcaca
tgtgttgttgttgatgtttctcatgaagatcctgaagtcaagttcaactggtacgttgatggtgttgaagttcataatgctaaga
caaagccaagagaagaacaatacaactctacttacagagttgtctctgttcttactgttctgcatcaagattggctgaatggtaa
ggaatacaagtgtaaggtctccaacaaagctcttccagctccaattgagaaaaccatttccaaagctaaaggtcaaccaagagaa
ccacaagtttacaccttgccaccatccagagatgaactgactaagaaccaagtctctctgacttgtctggttaaaggtttctatc
catctgatattgctgttgaatgggagtctaatggtcaaccagaaaacaactacaagactactcctcctgttctggattctgatgg
ttccttcttcctttactctaagcttactgttgataagtccagatggcaacaaggtaacgtcttctcatgttccgttatgcatgaa
gctttgcataaccattacactcagaagtctctttccctgtctccaggtaaataa
SEQ ID NO: 52
mh285L-1
cggaattc-gatgctgttatgactcaaaacccattgtctttgcctgtttctcttggtgatgaagcttctatttcttgtag
SEQ ID NO: 53
mh285L-2
agaaccagttcaagaaagtgttaccgttagagttttccaaagattgagaggatctacaagaaatagaagcttcat
SEQ ID NO: 54
mh285L-3
actttcttgaactggttctttcagaagccaggtcaatctccacaattgttgatttacagagtttctaacagattt
SEQ ID NO: 55
mh285L-4
caaagtgaaatcagtaccagaaccagaaccagaaaatctatctggaacaccagaaaatctgttagaaactctgta
SEQ ID NO: 56
mh285L-5
tctggtactgatttcactttgaagatttctagagttgaagctgaagatttgggtgtttacttctgtttgcaagttac
SEQ ID NO: 57
mh285L-6
caacagttctcttaatttccaaagtagtaccaccaccaaaagtgtatggaacatgagtaacttgcaaacagaagtaa
SEQ ID NO: 58
mh285L-7
tggaaattaagagaactgttgctgctccatctgtcttcatctttccaccatctgatgaacaattgaagtctggta
SEQ ID NO: 59
mh285L-8
tgaaccttagcttctcttgggtagaagttgttaagaagacaaacaacagaagcagtaccagacttcaattgttcat
SEQ ID NO: 60
mh285L-9
cccaagagaagctaaggttcagtggaaggttgataacgctttgcaatctggtaactctcaagaatctgttactgaa
SEQ ID NO: 61
mh285L-10
ccttagacaaagtcaaagtagaagacaaagagtaagtagaatccttagaatcttgttcagtaacagattcttgaga
SEQ ID NO: 62
mh285L-11
ctactttgactttgtctaaggctgattacgaaaagcataaggtttacgcttgtgaagttactcatcaaggtttgtc
SEQ ID NO: 63
mh285L-12
ggggtaccctaacattcacctctgttaaaggacttagtaactggagaagacaaaccttgatgagtaac
SEQ ID NO: 64
mh285H-1
cggaattc-gatattcaattgcaacaatctggtccaggtttggttaagccatctcaatctttgtctttgacttgttctg
SEQ ID NO: 65
mh285H-2
ggaaattgtctaatccagttccagttgtagttagtagtaatagagtaaccagtaacagaacaagtcaaagacaaag
SEQ ID NO: 66
mh285H-3
aactggattagacaatttccaggtaacaagttggaatggatgggttacattagatacgatggtacttctgaatac
SEQ ID NO: 67
mh285H-4
attggttcatagaagtatctctagtaatagaaactctgttcttcaaagatggggtgtattcagaagtaccatcgta
SEQ ID NO: 68
mh285H-5
gagatacttctatgaaccaattcttcttgagattgacttctgttactccagaagatactgctacttactactgtgc
SEQ ID NO: 69
mh285H-6
agtagaagcagaagaaacagtaacagaagtaccttgaccccagtaatccaatctagcacagtagtaagtagcagta
SEQ ID NO: 70
mh285H-7
ctgtttcttctgcttctactaagggtccatctgtttttccacttgctccatcttctaagtctacttctggtggta
SEQ ID NO: 71
mh285H-8
gaaacagtaactggttctggaaagtaatccttaaccaaacaacccaaagcagcagtaccaccagaagtagactta
SEQ ID NO: 72
mh285H-9
tccagaaccagttactgtttcttggaactctggtgctttgacttctggtgttcatacttttccagctgttttgcaa
SEQ ID NO: 73
mh285H-10
ccaaagaagaagatggaacagtaacaacagaagacaaagagtacaaaccagaagattgcaaaacagctggaaaagt
SEQ ID NO: 74
mh285H-11
ctgttccatcttcttctttgggtactcaaacttacatttgtaacgttaaccataagccatctaacactaaggttga
SEQ ID NO: 75
mh285H-12
tgtatgagttttatcacaagattttggttcaactctcttatcaaccttagtgttagatgg
SEQ ID NO: 76
Fc-1
5′gctgaaccaaaatcttgtgataaaactcatacatgtccaccatgtccagctcctgaacttctgggtggaccatcagtttt 3′
SEQ ID NO: 77
Fc-2
5′atgtgacttcaggagttctagaaatcataagggtatcctttggttttggtgggaacaagaaaactgatggtccacccaga 3′
SEQ ID NO: 78
Fc-3
5′ctagaacccctgaagtcacatgtgttgttgttgatgtttctcatgaagatcctgaagtcaagttcaactggtacgttgat 3′
SEQ ID NO: 79
Fc-4
5′taagtagagttgtattgttcttctcttggctttgtcttagcattatgaacttcaacaccatcaacgtaccagttgaactt 3′
SEQ ID NO: 80
Fc-5
5′agaacaatacaactctacttacagagttgtctctgttcttactgttctgcatcaagattggctgaatggtaaggaataca 3′
SEQ ID NO: 81
Fc-6
5′agctttggaaatggttttctcaattggagctggaagagctttgttggagaccttacacttgtattccttaccattcagcc 3′
SEQ ID NO: 82
Fc-7
5′gagaaaaccatttccaaagctaaaggtcaaccaagagaaccacaagtttacaccttgccaccatccagagatgaactgac 3′
SEQ ID NO: 83
Fc-8
5′cagcaatatcagatggatagaaacctttaaccagacaagtcagagagacttggttcttagtcagttcatctctggatggt 3′
SEQ ID NO: 84
Fc-9
5′tctatccatctgatattgctgttgaatgggagtctaatggtcaaccagaaaacaactacaagactactcctcctgttctg 3′
SEQ ID NO: 85
Fc-10
5′tgccatctggacttatcaacagtaagcttagagtaaaggaagaaggaaccatcagaatccagaacaggaggagtagtctt 3′
SEQ ID NO: 86
Fc-11
5′tgttgataagtccagatggcaacaaggtaacgtcttctcatgttccgttatgcatgaagctttgcataaccattacactc 3′
SEQ ID NO: 87
Fc-12
5′ ttatttacctggagacagggaaagagacttctgagtgtaatggttatgcaaag 3′
SEQ ID NO: 88
Fc/LP5
5′ ggggtaccttatttacctggagacagggaaagagacttct 3′
SEQ ID NO: 89
(human serum albumin, HSA)
atgaagtgggtaacctttatttcccttctttttctctttagctcggcttattccaggggtgtgtttcgtcgagatgcacacaaga
gtgaggttgctcatcggtttaaagatttgggagaagaaaatttcaaagccttggtgttgattgcctttgctcagtatcttcagca
gtgtccatttgaagatcatgtaaaattagtgaatgaagtaactgaatttgcaaaaacatgtgttgctgatgagtcagctgaaaat
tgtgacaaatcacttcataccctttttggagacaaattatgcacagttgcaactcttcgtgaaacctatggtgaaatggctgact
gctgtgcaaaacaagaacctgagagaaatgaatgcttcttgcaacacaaagatgacaacccaaacctcccccgattggtgagacc
agaggttgatgtgatgtgcactgcttttcatgacaatgaagagacatttttgaaaaaatacttatatgaaattgccagaagacat
ccttacttttatgccccggaactccttttctttgctaaaaggtataaagctgcttttacagaatgttgccaagctgctgataaag
ctgcctgcctgttgccaaagctcgatgaacttcgggatgaagggaaggcttcgtctgccaaacagagactcaagtgtgccagtct
ccaaaaatttggagaaagagctttcaaagcatgggcagtagctcgcctgagccagagatttcccaaagctgagtttgcagaagtt
tccaagttagtgacagatcttaccaaagtccacacggaatgctgccatggagatctgcttgaatgtgctgatgacagggcggacc
ttgccaagtatatctgtgaaaatcaagattcgatctccagtaaactgaaggaatgctgtgaaaaacctctgttggaaaaatccca
ctgcattgccgaagtggaaaatgatgagatgcctgctgacttgccttcattagctgctgattttgttgaaagtaaggatgtttgc
aaaaactatgctgaggcaaaggatgtcttcctgggcatgtttttgtatgaatatgcaagaaggcatcctgattactctgtcgtgc
tgctgctgagacttgccaagacatatgaaaccactctagagaagtgctgtgccgctgcagatcctcatgaatgctatgccaaagt
gttcgatgaatttaaacctcttgtggaagagcctcagaatttaatcaaacaaaattgtgagctttttgagcagcttggagagtac
aaattccagaatgcgctattagttcgttacaccaagaaagtaccccaagtgtcaactccaactcttgtagaggtctcaagaaacc
taggaaaagtgggcagcaaatgttgtaaacatcctgaagcaaaaagaatgccctgtgcagaag
SEQ ID NO: 90
(thrombin site)
ctcgagcccggcggcggcggcggccgcctggttcctcgtggcttcggtacc

1. A composition comprising a plurality of immunoglobulins, each immunoglobulin comprising at least one N-glycan attached thereto wherein the composition thereby comprises a plurality of N-glycans in which the predominant N-glycan consists essentially of Man₅GlcNAc₂.

2. The composition of claim 1, wherein greater than 50 mole percent of said plurality of N-glycans consists essentially of Man₅GlcNAc₂.

3. The composition of claim 1, wherein greater than 75 mole percent of said plurality of N-glycans consists essentially of Man₅GlcNAc₂.

4. The composition of claim 1, wherein greater than 90 mole percent of said plurality of N-glycans consists essentially of Man₅GlcNAc₂.

5. The composition of claim 1, wherein said Man₅GlcNAc₂ N-glycan is present at a level from about 5 mole percent to about 50 mole percent more than the next most predominant N-glycan structure of said plurality of N-glycans.

6. The composition of claim 1, wherein said immunoglobulins exhibit decreased binding affinity for an FcγRIIb receptor.

7. The composition of claim 1, wherein said immunoglobulins exhibit increased binding affinity for an FcγRIII receptor.

8. The composition of claim 7, wherein said FcγRIII receptor is a FcγRIIIa receptor.

9. The composition of claim 7, wherein said FcγRIII receptor is a FcγRIIIb receptor.

10. The composition of claim 1, wherein said immunoglobulins exhibit increased antibody-dependent cellular cytotoxicity (ADCC) activity.

11. The composition of claim 1, wherein said immunoglobulins are essentially free of fucose.

12. The composition of claim 1, wherein said immunoglobulins lack fucose.

13. The composition of claim 1, wherein said immunoglobulins bind to an antigen selected from the group consisting of growth factors, FGFR, EGFR, VEGF, leukocyte antigens, CD20, CD33, cytokines, TNF-α and TNF-β.

14. The composition of claim 1, wherein said immunoglobulins comprise an Fc region selected from the group consisting of an IgG1, IgG2, IgG3 and IgG4 region.

15. A pharmaceutical composition comprising the composition of claim 1 and a pharmaceutically acceptable carrier.

16. The pharmaceutical composition of claim 15, wherein said immunoglobulins are essentially free of fucose.

17. The pharmaceutical composition of claim 15, wherein said immunoglobulins lack fucose.

18. The pharmaceutical composition of claim 15, wherein said immunoglobulins comprise an antibody which binds to an antigen selected from the group consisting of growth factors, FGFR, EGFR, VEGF, leukocyte antigens, CD20, CD33, cytokines, TNF-αand TNF-β.

19. The pharmaceutical composition of claim 15, wherein said immunoglobulins comprise an Fc region selected from the group consisting of an IgG1, IgG2, IgG3 and IgG4 region.

20. A kit comprising the composition of claim 1.

21. A eukaryotic host cell comprising an exogenous gene encoding an immunoglobulin or fragment thereof, said eukaryotic host cell engineered or selected to express said immunoglobulin or fragment thereof, thereby producing a composition comprising a plurality of immunoglobulins, each immunoglobulin comprising at least one N-glycan attached thereto wherein the composition thereby comprises a plurality of N-glycans in which the predominant N-glycan consists essentially of Man₅GlcNAc₂.

22. The host cell of claim 21 wherein the host cell is a lower eukaryotic host cell.

23. A method for producing in a eukaryotic host a composition comprising a plurality of immunoglobulins, each immunoglobulin comprising at least one N-glycan attached thereto wherein the composition thereby comprises a plurality of N-glycans in which the predominant N-glycan consists essentially of Man₅GlcNAc₂.

24. The method of claim 23 wherein the host cell is a lower eukaryotic host cell.


SEQUENCE LISTING (90 sequences)

SEQ ID NO: 1 - DNA, 642 bp, Artificial Sequence
  Description of Artificial Sequence Mouse/human chimeric IgG1 light chain
caaatcgtcttgtctcaatccccagctattttgtctgcttcccctggagagaaggtcaccatgacttgtagagcctcttcctctgtctcttacattcactggttccagcaaaagccaggttcctctccaaagccatggatctacgctacttccaacttggcttccggtgttccagttagattctctggttctggttccggtacctcctactctcttaccatctccagagttgaagccgaggacgctgctacttactactgtcagcaatggacttctaacccaccaactttcggtggtggtaccaaattggagattaagagaactgttgctgctccatccgttttcattttcccaccatccgacgaacaattgaagtctggtacagcttccgttgtttgtttgttgaacaacttctacccaagagaggctaaggttcagtggaaggttgacaacgctttgcaatccggtaactcccaagaatccgttactgagcaggattctaaggattccacttactccttgtcctccactttgactttgtccaaggctgattacgagaagcacaaggtttacgcttgtgaggttacacatcagggtttgtcctccccagttactaagtccttcaacagaggagagtgttaa

SEQ ID NO: 2 - DNA, 1354 bp, Artificial Sequence
  Description of Artificial Sequence Mouse/human chimeric IgG1 heavy chain
caagtccagttgcaacagcctggtgccgagttggtcaagccaggtgcttctgttaagatgtcctgtaaggcttctggttacactttcacctcctacaacatgcactgggtcaagcaaactccaggtagaggtttggagtggttggtgccatctacccaggtaacggtgacacttcttacaaccaaaaattcaagggaaaggctactcttaccgctgataagtcctcttccaccgcctacatgcaattgtcttccttgacttctgaagattctgctgtttactactgtgctagatccacctactacggtggagactggtacttcaacgtttggggtgctggtaccactgtcaccgtttccgctgcttctactaagggaccatccgtttttccattggctccatcctctaagtctacttccggtggtactgctgctttgggatgtttggttaaggactacttcccagagcctgttactgtttcttggaactccggtgctttgacttctggtgttcacactttcccagctgttttgcaatcttccggtttgtactccttgtcctccgttgttactgttccatcctcttccttgggtactcagacttacatctgtaacgttaaccacaagccatccaacactaaggttgacaagaaggctgagccaaagtcctgtgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaggagcagtacaactccacttacagagttgtttccgttttgactgttttgcaccaggattggttgaacggaaaggagtacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaaaggattctacccatccgacattgctgttgagtgggaatctaacggtcaaccagagaacaactacaagactactccaccagttttggattctgacggttccttcttcttgtactccaagttgactgttgacaagtccagatggaacagggtaacgttttctcctgttccgttatgcatgaggctttgcacaaccactacactcaaaagtccttgtctttgtccccaggtaagtaa

SEQ ID NO: 3 - DNA, 324 bp, Homo sapiens
agaactgttgctgctccatccgttttcattttcccaccatccgacgaacaattgaagtctggtacagcttccgttgtttgtttgttgaacaacttctacccaagagaggctaaggttcagtggaaggttgacaacgctttgcaatccggtaactcccaagaatccgttactgagcaggattctaaggattccacttactccttgtcctccactttgactttgtccaaggctgattacgagaagcacaaggtttacgcttgtgaggttacacatcagggtttgtcctccccagttactaagtccttcaacagaggagagtgttaa

SEQ ID NO: 4 - DNA, 989 bp, Homo sapiens
tctactaagggaccatccgtttttccattggctccatcctctaagtctacttccggtggtactgctgctttgggatgtttggttaaggactacttcccagagcctgttactgtttcttggaactccggtgctttgacttctggtgttcacactttcccagctgttttgcaatcttccggtttgtactccttgtcctccgttgttactgttccatcctcttccttgggtactcagacttacatctgtaacgttaaccacaagccatccaacactaaggttgacaagaaggctgagccaaagtcctgtgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaggagcagtacaactccacttacagagttgtttccgttttgactgttttgcaccaggattggttgaacggaaaggagtacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaaaggattctacccatccgacattgctgttgagtgggaatctaacggtcaaccagagaacaactacaagactactccaccagttttggattctgacggttccttcttcttgtactccaagttgactgttgacaagtccagatggaacagggtaacgttttctcctgttccgttatgcatgaggctttgcacaaccactacactcaaaagtccttgtctttgtccccaggtaagtaa

SEQ ID NO: 5 - DNA, 45 bp, Artificial Sequence
  Description of Artificial Sequence Synthetic oligonucleotide
aggagtcgtattcaaatcgtcttgtctcaatccccagctattttg

SEQ ID NO: 6 - DNA, 45 bp, Artificial Sequence
  Description of Artificial Sequence Synthetic oligonucleotide
tctgcttcccctggagagaaggtcaccatgacttgtagagcctct

SEQ ID NO: 7 - DNA, 45 bp, Artificial Sequence
  Description of Artificial Sequence Synthetic oligonucleotide
tcctctgtctcttacattcactggttccagcaaaagccaggttcc

SEQ ID NO: 8 - DNA, 45 bp, Artificial Sequence
  Description of Artificial Sequence Synthetic oligonucleotide
tctccaaagccatggatctacgctacttccaacttggcttccggt

SEQ ID NO: 9 - DNA, 45 bp, Artificial Sequence
  Description of Artificial Sequence Synthetic oligonucleotide
gttccagttagattctctggttctggttccggtacctcctactct

SEQ ID NO: 10 - DNA, 45 bp, Artificial Sequence
  Description of Artificial Sequence Synthetic oligonucleotide
cttaccatctccagagttgaagccgaggacgctgctacttactac

SEQ ID NO: 11 - DNA, 45 bp, Artificial Sequence
  Description of Artificial Sequence Synthetic oligonucleotide
tgtcagcaatggacttctaacccaccaactttcggtggtggtacc

SEQ ID NO: 12 - DNA, 36 bp, Artificial Sequence
  Description of Artificial Sequence Synthetic oligonucleotide
aaattggagattaagagaactgttgctgctccatcc

SEQ ID NO: 13 - DNA, 45 bp, Artificial Sequence
  Description of Artificial Sequence Synthetic oligonucleotide
caacagttctcttaatctccaatttggtaccaccaccgaaagttg

SEQ ID NO: 14 - DNA, 45 bp, Artificial Sequence
  Description of Artificial Sequence Synthetic oligonucleotide
gtgggttagaagtccattgctgacagtagtaagtagcagcgtcct

SEQ ID NO: 15 - DNA, 45 bp, Artificial Sequence
  Description of Artificial Sequence Synthetic oligonucleotide
cggcttcaactctggagatggtaagagagtaggaggtaccggaac

SEQ ID NO: 16 - DNA, 44 bp, Artificial Sequence
  Description of Artificial Sequence Synthetic oligonucleotide
agaaccagagaatctaactggaacaccggaagccaagttggaag

SEQ ID NO: 17 - DNA, 45 bp, Artificial Sequence
  Description of Artificial Sequence Synthetic oligonucleotide
tagcgtagatccatggctttggagaggaacctggcttttgctgga

SEQ ID NO: 18 - DNA, 44 bp, Artificial Sequence
  Description of Artificial Sequence Synthetic oligonucleotide
ccagtgaatgtaagagacagaggaagaggctctacaagtcatgg

SEQ ID NO: 19 - DNA, 45 bp, Artificial Sequence
  Description of Artificial Sequence Synthetic oligonucleotide
tgaccttctctccaggggaagcagacaaaatagctggggattgag

SEQ ID NO: 20 - DNA, 21 bp, Artificial Sequence
  Description of Artificial Sequence Synthetic oligonucleotide
aggagtcgtattcaaatcgtc

SEQ ID NO: 21 - DNA, 21 bp, Artificial Sequence
  Description of Artificial Sequence Synthetic oligonucleotide
agaactgttgctgctccatcc

SEQ ID NO: 22 - DNA, 20 bp, Artificial Sequence
  Description of Artificial Sequence Synthetic oligonucleotide
ggatggagcagcaacagttc

SEQ ID NO: 23 - DNA, 30 bp, Artificial Sequence
  Description of Artificial Sequence Synthetic oligonucleotide
ctggtaccttaacactctcctctgttgaag

SEQ ID NO: 24 - DNA, 45 bp, Artificial Sequence
  Description of Artificial Sequence Synthetic oligonucleotide
aggagtcgtattcaagtccagttgcaacagcctggtgccgagttg

SEQ ID NO: 25 - DNA, 45 bp, Artificial Sequence
  Description of Artificial Sequence Synthetic oligonucleotide
gtcaagccaggtgcttctgttaagatgtcctgtaaggcttctggt

SEQ ID NO: 26 - DNA, 45 bp, Artificial Sequence
  Description of Artificial Sequence Synthetic oligonucleotide
tacactttcacctcctacaacatgcactgggtcaagcaaactcca

SEQ ID NO: 27 - DNA, 45 bp, Artificial Sequence
  Description of Artificial Sequence Synthetic oligonucleotide
ggtagaggtttggagtggattggtgccatctacccaggtaacggt

SEQ ID NO: 28 - DNA, 45 bp, Artificial Sequence
  Description of Artificial Sequence Synthetic oligonucleotide
gacacttcttacaaccaaaaattcaagggaaaggctactcttacc

SEQ ID NO: 29 - DNA, 45 bp, Artificial Sequence
  Description of Artificial Sequence Synthetic oligonucleotide
gctgataagtcctcttccaccgcctacatgcaattgtcttccttg

SEQ ID NO: 30 - DNA, 45 bp, Artificial Sequence
  Description of Artificial Sequence Synthetic oligonucleotide
acttctgaagactctgctgtttactactgtgctagatccacctac

SEQ ID NO: 31 - DNA, 45 bp, Artificial Sequence
  Description of Artificial Sequence Synthetic oligonucleotide
tacggtggagactggtacttcaacgtttggggtgctggtaccact

SEQ ID NO: 32 - DNA, 36 bp, Artificial Sequence
  Description of Artificial Sequence Synthetic oligonucleotide
gtcaccgtttccgctgcttctactaagggaccatcc

SEQ ID NO: 33 - DNA, 45 bp, Artificial Sequence
  Description of Artificial Sequence Synthetic oligonucleotide
tagtagaagcagcggaaacggtgacagtggtaccagcaccccaaa

SEQ ID NO: 34 - DNA, 44 bp, Artificial Sequence
  Description of Artificial Sequence Synthetic oligonucleotide
cgttgaagtaccagtctccaccgtagtaggtggatctagcacag

SEQ ID NO: 35 - DNA, 45 bp, Artificial Sequence
  Description of Artificial Sequence Synthetic oligonucleotide
agtaaacagcagagtcttcagaagtcaaggaagacaattgcatgt

SEQ ID NO: 36 - DNA, 45 bp, Artificial Sequence
  Description of Artificial Sequence Synthetic oligonucleotide
aggcggtggaagaggacttatcagcggtaagagtagcctttccct

SEQ ID NO: 37 - DNA, 45 bp, Artificial Sequence
  Description of Artificial Sequence Synthetic oligonucleotide
tgaatttttggttgtaagaagtgtcaccgttacctgggtagatgg

SEQ ID NO: 38 - DNA, 45 bp, Artificial Sequence
  Description of Artificial Sequence Synthetic oligonucleotide
caccaatccactccaaacctctacctggagtttgcttgacccagt

SEQ ID NO: 39 - DNA, 45 bp, Artificial Sequence
  Description of Artificial Sequence Synthetic oligonucleotide
gcatgttgtaggaggtgaaagtgtaaccagaagccttacaggaca

SEQ ID NO: 40 - DNA, 45 bp, Artificial Sequence
  Description of Artificial Sequence Synthetic oligonucleotide
tcttaacagaagcacctggcttgaccaactcggcaccaggctgtt

SEQ ID NO: 41 - DNA, 21 bp, Artificial Sequence
  Description of Artificial Sequence Synthetic oligonucleotide
aggagtcgtattcaagtccag

SEQ ID NO: 42 - DNA, 21 bp, Artificial Sequence
  Description of Artificial Sequence Synthetic oligonucleotide
gcttctactaagggaccatcc

SEQ ID NO: 43 - DNA, 21 bp, Artificial Sequence
  Description of Artificial Sequence Synthetic oligonucleotide
ggatggtcccttagtagaagc

SEQ ID NO: 44 - DNA, 28 bp, Artificial Sequence
  Description of Artificial Sequence Synthetic oligonucleotide
ctggtattacttacctggggacaaagac

SEQ ID NO: 45 - DNA, 105 bp, Artificial Sequence
  Description of Artificial Sequence Synthetic oligonucleotide
gaattcgaaacgatgctgtcgttaaaaccatcttggctgactttggcggcattaatgtatgccatgctattggtcgtagtgccatttgctaaacctgttagagct

SEQ ID NO: 46 - DNA, 70 bp, Artificial Sequence
  Description of Artificial Sequence Synthetic oligonucleotide
aattcgaaacgatgctgtctttgaagccatcttggcttactttggctgctttgatgtacgctatgctttt

SEQ ID NO: 47 - DNA, 41 bp, Artificial Sequence
  Description of Artificial Sequence Synthetic oligonucleotide
ccaaagtaagccaagatggcttcaaagacagcatcgtttcg

SEQ ID NO: 48 - DNA, 34 bp, Artificial Sequence
  Description of Artificial Sequence Synthetic oligonucleotide
ggttgttgttccatttgctaagccagttagagct

SEQ ID NO: 49 - DNA, 59 bp, Artificial Sequence
  Description of Artificial Sequence Synthetic oligonucleotide
agctctaactggcttagcaaatggaacaacaaccaaaagcatagcgtacatcaaagcag

SEQ ID NO: 50 - DNA, 660 bp, Mus musculus
gatgctgttatgactcaaaacccattgtctttgcctgtttctcttggtgatgaagcttctatttcttgtagatcctctcaatctttggaaaactctaacggtaacactttcttgaactggttctttcagaagccaggtcaatctccacaattgttgatttacagagtttctaacagattttctggtgttccagatagattttctggttctggttctggtactgatttcactttgaagatttctagagttgaagctgaagatttgggtgtttacttctgtttgcaagttactcatgttccatacacttttggtggtggtactactttggaaattaagagaactgttgctgctccatctgtcttcatctttccaccatctgatgaacaattgaagtctggtactgcttctgttgtttgtcttcttaacaacttctacccaagagaagctaaggttcagtggaaggttgataacgctttgcaatctggtaactctcaagaatctgttactgaacaagattctaaggattctacttactctttgtcttctactttgactttgtctaaggctgattacgaaaagcataaggtttacgcttgtgaagttactcatcaaggtttgtcttctccagttactaagtcctttaacagaggtgaatgttag

SEQ ID NO: 51 - DNA, 1329 bp, Mus musculus
gatattcaattgcaacaatctggtccaggtttggttaagccatctcaatctttgtctttgacttgttctgttactggttactctattactactaactacaactggaactggattagacaatttccaggtaacaagttggaatggatgggttacattagatacgatggtacttctgaatacaccccatctttgaagaacagagtttctattactagagatacttctatgaaccaattcttcttgagattgacttctgttactccagaagatactgctacttactactgtgctagattggattactggggtcaaggtacttctgttactgtttcttctgcttctactaagggtccatctgtttttccacttgctccatcttctaagtctacttctggtggtactgctgctttgggttgtttggttaaggattactttccagaaccagttactgtttcttggaactctggtgctttgacttctggtgttcatacttttccagctgttttgcaatcttctggtttgtactctttgtcttctgttgttactgttccatcttcttctttgggtactcaaacttacatttgtaacgttaaccataagccatctaacactaaggttgataagagagttgaaccaaaatcttgtgataaaactcatacatgtccaccatgtccagctcctgaacttctgggtggaccatcagttttcttgttcccaccaaaaccaaaggatacccttatgatttctagaactcctgaagtcacatgtgttgttgttgatgtttctcatgaagatcctgaagtcaagttcaactggtacgttgatggtgttgaagttcataatgctaagacaaagccaagagaagaacaatacaactctacttacagagttgtctctgttcttactgttctgcatcaagattggctgaatggtaaggaatacaagtgtaaggtctccaacaaagctcttccagctccaattgagaaaaccatttccaaagctaaaggtcaaccaagagaaccacaagtttacaccttgccaccatccagagatgaactgactaagaaccaagtctctctgacttgtctggttaaaggtttctatccatctgatattgctgttgaatgggagtctaatggtcaaccagaaaacaactacaagactactcctcctgttctggattctgatggttccttcttcctttactctaagcttactgttgataagtccagatggcaacaaggtaacgtcttctcatgttccgttatgcatgaagctttgcataaccattacactcagaagtctctttccctgtctccaggtaaataa

SEQ ID NO: 52 - DNA, 71 bp, Artificial Sequence
  Description of Artificial Sequence Synthetic oligonucleotide
gatgctgttatgactcaaaacccattgtctttgcctgtttctcttggtgatgaagcttctatttcttgtag

SEQ ID NO: 53 - DNA, 75 bp, Artificial Sequence
  Description of Artificial Sequence Synthetic oligonucleotide
agaaccagttcaagaaagtgttaccgttagagttttccaaagattgagaggatctacaagaaatagaagcttcat

SEQ ID NO: 54 - DNA, 75 bp, Artificial Sequence
  Description of Artificial Sequence Synthetic oligonucleotide
actttcttgaactggttctttcagaagccaggtcaatctccacaattgttgatttacagagtttctaacagattt

SEQ ID NO: 55 - DNA, 75 bp, Artificial Sequence
  Description of Artificial Sequence Synthetic oligonucleotide
caaagtgaaatcagtaccagaaccagaaccagaaaatctatctggaacaccagaaaatctgttagaaactctgta

SEQ ID NO: 56 - DNA, 77 bp, Artificial Sequence
  Description of Artificial Sequence Synthetic oligonucleotide
tctggtactgatttcactttgaagatttctagagttgaagctgaagatttgggtgtttacttctgtttgcaagttac

SEQ ID NO: 57 - DNA, 77 bp, Artificial Sequence
  Description of Artificial Sequence Synthetic oligonucleotide
caacagttctcttaatttccaaagtagtaccaccaccaaaagtgtatggaacatgagtaacttgcaaacagaagtaa

SEQ ID NO: 58 - DNA, 75 bp, Artificial Sequence
  Description of Artificial Sequence Synthetic oligonucleotide
tggaaattaagagaactgttgctgctccatctgtcttcatctttccaccatctgatgaacaattgaagtctggta

SEQ ID NO: 59 - DNA, 76 bp, Artificial Sequence
  Description of Artificial Sequence Synthetic oligonucleotide
tgaaccttagcttctcttgggtagaagttgttaagaagacaaacaacagaagcagtaccagacttcaattgttcat

SEQ ID NO: 60 - DNA, 76 bp, Artificial Sequence
  Description of Artificial Sequence Synthetic oligonucleotide
cccaagagaagctaaggttcagtggaaggttgataacgctttgcaatctggtaactctcaagaatctgttactgaa

SEQ ID NO: 61 - DNA, 76 bp, Artificial Sequence
  Description of Artificial Sequence Synthetic oligonucleotide
ccttagacaaagtcaaagtagaagacaaagagtaagtagaatccttagaatcttgttcagtaacagattcttgaga

SEQ ID NO: 62 - DNA, 76 bp, Artificial Sequence
  Description of Artificial Sequence Synthetic oligonucleotide
ctactttgactttgtctaaggctgattacgaaaagcataaggtttacgcttgtgaagttactcatcaaggtttgtc

SEQ ID NO: 63 - DNA, 68 bp, Artificial Sequence
  Description of Artificial Sequence Synthetic oligonucleotide
ggggtaccctaacattcacctctgttaaaggacttagtaactggagaagacaaaccttgatgagtaac

SEQ ID NO: 64 - DNA, 78 bp, Artificial Sequence
  Description of Artificial Sequence Synthetic oligonucleotide
cggaattcgatattcaattgcaacaatctggtccaggtttggttaagccatctcaatctttgtctttgacttgttctg

SEQ ID NO: 65 - DNA, 76 bp, Artificial Sequence
  Description of Artificial Sequence Synthetic oligonucleotide
ggaaattgtctaatccagttccagttgtagttagtagtaatagagtaaccagtaacagaacaagtcaaagacaaag

SEQ ID NO: 66 - DNA, 75 bp, Artificial Sequence
  Description of Artificial Sequence Synthetic oligonucleotide
aactggattagacaatttccaggtaacaagttggaatggatgggttacattagatacgatggtacttctgaatac

SEQ ID NO: 67 - DNA, 76 bp, Artificial Sequence
  Description of Artificial Sequence Synthetic oligonucleotide
attggttcatagaagtatctctagtaatagaaactctgttcttcaaagatggggtgtattcagaagtaccatcgta

SEQ ID NO: 68 - DNA, 76 bp, Artificial Sequence
  Description of Artificial Sequence Synthetic oligonucleotide
gagatacttctatgaaccaattcttcttgagattgacttctgttactccagaagatactgctacttactactgtgc

SEQ ID NO: 69 - DNA, 76 bp, Artificial Sequence
  Description of Artificial Sequence Synthetic oligonucleotide
agtagaagcagaagaaacagtaacagaagtaccttgaccccagtaatccaatctagcacagtagtaagtagcagta

SEQ ID NO: 70 - DNA, 75 bp, Artificial Sequence
  Description of Artificial Sequence Synthetic oligonucleotide
ctgtttcttctgcttctactaagggtccatctgtttttccacttgctccatcttctaagtctacttctggtggta

SEQ ID NO: 71 - DNA, 75 bp, Artificial Sequence
  Description of Artificial Sequence Synthetic oligonucleotide
gaaacagtaactggttctggaaagtaatccttaaccaaacaacccaaagcagcagtaccaccagaagtagactta

SEQ ID NO: 72 - DNA, 76 bp, Artificial Sequence
  Description of Artificial Sequence Synthetic oligonucleotide
tccagaaccagttactgtttcttggaactctggtgctttgacttctggtgttcatacttttccagctgttttgcaa

SEQ ID NO: 73 - DNA, 76 bp, Artificial Sequence
  Description of Artificial Sequence Synthetic oligonucleotide
ccaaagaagaagatggaacagtaacaacagaagacaaagagtacaaaccagaagattgcaaaacagctggaaaagt

SEQ ID NO: 74 - DNA, 76 bp, Artificial Sequence
  Description of Artificial Sequence Synthetic oligonucleotide
ctgttccatcttcttctttgggtactcaaacttacatttgtaacgttaaccataagccatctaacactaaggttga

SEQ ID NO: 75 - DNA, 60 bp, Artificial Sequence
  Description of Artificial Sequence Synthetic oligonucleotide
tgtatgagttttatcacaagattttggttcaactctcttatcaaccttagtgttagatgg

SEQ ID NO: 76 - DNA, 80 bp, Artificial Sequence
  Description of Artificial Sequence Synthetic oligonucleotide
gctgaaccaaaatcttgtgataaaactcatacatgtccaccatgtccagctcctgaacttctgggtggaccatcagtttt

SEQ ID NO: 77 - DNA, 80 bp, Artificial Sequence
  Description of Artificial Sequence Synthetic oligonucleotide
atgtgacttcaggagttctagaaatcataagggtatcctttggttttggtgggaacaagaaaactgatggtccacccaga

SEQ ID NO: 78 - DNA, 80 bp, Artificial Sequence
  Description of Artificial Sequence Synthetic oligonucleotide
ctagaacccctgaagtcacatgtgttgttgttgatgtttctcatgaagatcctgaagtcaagttcaactggtacgttgat

SEQ ID NO: 79 - DNA, 80 bp, Artificial Sequence
  Description of Artificial Sequence Synthetic oligonucleotide
taagtagagttgtattgttcttctcttggctttgtcttagcattatgaacttcaacaccatcaacgtaccagttgaactt

SEQ ID NO: 80 - DNA, 80 bp, Artificial Sequence
  Description of Artificial Sequence Synthetic oligonucleotide
agaacaatacaactctacttacagagttgtctctgttcttactgttctgcatcaagattggctgaatggtaaggaataca

SEQ ID NO: 81 - DNA, 80 bp, Artificial Sequence
  Description of Artificial Sequence Synthetic oligonucleotide
agctttggaaatggttttctcaattggagctggaagagctttgttggagaccttacacttgtattccttaccattcagcc

SEQ ID NO: 82 - DNA, 80 bp, Artificial Sequence
  Description of Artificial Sequence Synthetic oligonucleotide
gagaaaaccatttccaaagctaaaggtcaaccaagagaaccacaagtttacaccttgccaccatccagagatgaactgac

SEQ ID NO: 83 - DNA, 80 bp, Artificial Sequence
  Description of Artificial Sequence Synthetic oligonucleotide
cagcaatatcagatggatagaaacctttaaccagacaagtcagagagacttggttcttagtcagttcatctctggatggt

SEQ ID NO: 84 - DNA, 80 bp, Artificial Sequence
  Description of Artificial Sequence Synthetic oligonucleotide
tctatccatctgatattgctgttgaatgggagtctaatggtcaaccagaaaacaactacaagactactcctcctgttctg

SEQ ID NO: 85 - DNA, 80 bp, Artificial Sequence
  Description of Artificial Sequence Synthetic oligonucleotide
tgccatctggacttatcaacagtaagcttagagtaaaggaagaaggaaccatcagaatccagaacaggaggagtagtctt

SEQ ID NO: 86 - DNA, 80 bp, Artificial Sequence
  Description of Artificial Sequence Synthetic oligonucleotide
tgttgataagtccagatggcaacaaggtaacgtcttctcatgttccgttatgcatgaagctttgcataaccattacactc

SEQ ID NO: 87 - DNA, 53 bp, Artificial Sequence
  Description of Artificial Sequence Synthetic oligonucleotide
ttatttacctggagacagggaaagagacttctgagtgtaatggttatgcaaag

SEQ ID NO: 88 - DNA, 40 bp, Artificial Sequence
  Description of Artificial Sequence Synthetic oligonucleotide
ggggtaccttatttacctggagacagggaaagagacttct

SEQ ID NO: 89 - DNA, 1423 bp, Homo sapiens
atgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaag

SEQ ID NO: 90 - DNA, 51 bp, Artificial Sequence
  Description of Artificial Sequence Synthetic oligonucleotide
ctcgagcccggcggcggcggcggccgcctggttcctcgtggcttcggtacc